(19) United States
(12) Patent Application Publication
(10) Pub. No.: US 2009/0227938 A1
(43) Pub. Date: Sep. 10, 2009

(54) Wound Closure Devices, Methods of Use, and Kits

(75) Inventors: Rainer Fasching, Mill Valley, CA (US); WonHyoung Ryu, Mountain View, CA (US); Darius Moshfeghi, Stanford, CA (US)

Correspondence Address: WILSON SONSINI GOODRICH & ROSATI, 650 PAGE MILL ROAD, PALO ALTO, CA, 94304-1050, US

(73) Assignee: InSitu Therapeutics, Inc., Mill Valley, CA (US)

(21) Appl. No.: 12/247,003

(22) Filed: Oct. 7, 2008

Related U.S. Application Data
(60) Provisional application No. 61/034,108, filed on May 15, 2008.
(60) Provisional application No. 61/034,110, filed on Mar. 5, 2008.
(60) Provisional application No. 61/040,500, filed on Mar. 28, 2008.

(51) Int. Cl. A61F 9/007

(52) U.S. Cl. 604/57; 606/213

(57) ABSTRACT

Provided herein is a wound closure device comprising a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening. The wound closure device can be used in cases where ocular surgery has been preformed.

### CROSS-REFERENCE

This application claims the benefit of U.S. Provisional Application No. 61/034,108, filed Mar. 5, 2008, and U.S. Provisional Application No. 61/034,110, filed Mar. 5, 2008, and 61/040,500, filed March 28, 2008, which applications are incorporated herein by reference in their entireties.

### BACKGROUND OF THE INVENTION

Surgery is constantly developing less and less invasive surgical techniques such as endoscopic based procedures in order to minimize the trauma inflicted upon a patient during surgery and minimize the recovery time to overcome the trauma of surgery. A wound can include surgical incisions as well as wounds caused by accidental trauma or disease. Wound sites generated inside the body or tissue damage are often not accessible and cannot be sufficiently treated or closed. Often open surgery must be performed to close and repair the wound sites. Open surgery can cause significant additional tissue damage and longer recovery time. Therefore a considerable body of literature is devoted to methods for improving wound closure for minimally invasive procedures, or methods for improving tissue damage inside the body or tissue damage covered by additional tissue layers such as endoscopic based procedure where the wound sites are not accessible.

Some wound plugs can also be used to deliver a medication to a wound. Topical administration of medications often fails to provide therapeutic levels in the vitreous cavity or posterior segment of the eye. There are significant barriers to solute flux in the corneal epithelium and the topical drops are rapidly lost as the result of drainage and tear fluid turnover. Drugs can be delivered by frequent injections, but it is not clinically and practically adequate for chronic diseases that can sometimes require multiple weekly administrations over months or years. In addition, the multiple intraocular injections can lead to an increased likelihood of complications such as vitreous hemorrhages, retinal detachment, and endophthalmitis. Systemic administration of medication is also very limited to the intraocular diseases due to the presence of blood-ocular barrier (Velez et al 1999, Geroki et al 2000).

In order to overcome these difficulties of intraocular administration, U.S. Pat. Nos. 5,443,505 and 5,824,072 describe a method of preparing and surgically introducing a drug delivery implant into avascular suprachoroidal space and pars plana to deliver antitumor agents and bacterial agents. The implant is prepared by combining a physiologically active therapeutic agent in a pharmacologically acceptable biocompatible polymer. The implant is surgically introduced extrinsic to the vitreous and anchored in the avascular implantation site. The pharmacologically active agent diffuses from the implant into the vitreous space. As another example, U.S. Pat. No. 6,964,781 describes a sustained release drug delivery device comprising a drug core, a unitary cup, and a prefabricated permeable plug. The device is intended to be surgically implanted to the vitreous of the eye, under the retina, and onto the sclera. U.S. Pat. No. 6,719,750 describes a coil shaped device that delivers therapeutic agents into the patient eye.

The advent of transconjunctival, sutureless, trochar-based vitrectomy has evolved from 20-gauge based instrumentation to the present offerings of 20-, 23-, 25-, and 27-gauge “suture-less” vitrectomy setups. The benefit of transconjunctival, trochar-based vitrectomy are: 1) surgical efficiency, 2) comfort, 3) decreased duration of surgery, 4) faster healing, 5) improved cosmesis, and 6) cost-savings. Limitations include hypotony, wound leak, loss of volume, and endophthalmitis (infection inside the eye). These limitations are related to lack of closure of the sclerotomy site. Despite anatomic attempts to limit wound gape (e.g. beveled wound construction, temporary displacement of the conjunctiva) the rate of endophthalmitis has been reported to be 12 times higher than with conventional 20-gauge sutured surgery. This rate of endophthalmitis is directly linked to the open wound—a gaped wound in conjunction with a pressure differential from inside the eye to outside the eye promotes intraocular inoculation from the normal conjunctival flora, leading to an endophthalmitis in a significant number of patients.

U.S. Pat. No. 5,707,643 describes a biodegradable scleral plug system. The plug is implanted through open wound from vitreous surgery and releases drugs by the degradation of the polymer. US Pat. App. No. 2005/0148948 describes a method (“Sutureless ophthalmic drug delivery system and method”) of using transconjunctival entry alignment device for insertion of a drug delivery device into the eye. While these inventions involve minimally invasive implantation of the drug delivery devices, they are limited in technical issues like the following. The onset of drug release from the biodegradable plug indicates the plug starts losing its physical integrity. On the other hand, in order to guarantee its function as a plug, the onset of drug release from the biodegradable plug has to be sustained. The sutureless drug delivery system allows minimally invasive application of drug delivery device into the vitreous space of the eye. However, the application still needs aid of tools to deliver and anchor the device to a target location within the eye. This requires very delicate and careful application processes and may need even longer time to finish the implantation. The elongated implantation procedure may cause surgical trauma as well.

Similarly, a transitory or chronic hypotony state (low pressure in the eye), predisposes to suprachoroidal hemorrhage and choroidal effusions. These, in addition to being painful, are vision limiting and can predispose to retinal detachment. They may warrant another trip to the operating room to perform wound reconstruction and closure, as well as to address the secondary complications (suprachoroidal hemorrhage, flat anterior chamber, retinal detachment, etc.)

Sutures have historically served to limit the complications listed above. However, suturing following a transconjunctival, trochar-based vitrectomy eliminates all of the benefits of the system. The reason for this is that the conjunctiva is the tissue that is most likely to bleed, cause discomfort, and result in poor cosmetics. Closure with suture increases the duration of the surgery and decreases the surgical efficiency.

Ideally, a transconjunctival closure of the sclerotomy sites would retain all of the benefits of a transconjunctival, sutureless, trochar-based vitrectomy system while eliminating the likelihood of significant rates of endophthalmitis, hypotony, wound leak, volume loss, and anatomic distortion. Additionally, such a transconjunctival wound closure system offers the possibility of serving as a reservoir of medication to decrease postoperative inflammation and reduce the chance of infection.

In light of the above, it would be desirable to provide a wound closure system that overcomes some of the above problems.

### SUMMARY OF THE INVENTION

Provided herein is a wound closure device comprising a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening. The wound closure device can be transition between a first configuration and the second configuration after exposure to one or more of an aqueous medium, change in temperature, a chemical environment, pH, ion strength, salt concentration, or light. The wound closure device can be a biocompatible material. The biocompatible material can be selected from at least one of a compressible material, temperature dependent material, shape memory material, a swellable material, and an expandable material. Additionally, the wound closure device can comprise an anchor adaptable to prevent removal of the wound closure device from a wound. The anchor can be a physical feature or a change in the external surface of the device that causes the device to anchor into the wound. The wound closure device can comprise a handle adaptable to insert the wound closure device in a wound. In some embodiments, the wound closure device is adaptable to be cut. Furthermore, the device can comprise markers along the length of the wound closure device to indicate depth of insertion of the device and to facilitate cutting of the device. Additionally, the wound closure device can comprise a drug delivery element. The wound closure device can be induced into the first configuration using at least one of a physical force, a chemical force, or a mechanical force. The wound closure device can be inserted into a wound using a device applicator where the device is a pre-cut device. Alternatively, the device can be cut by the applicator after being inserted into the wound. The wound closure device applicator can insert the wound closure device into the wound through a cannula. The device can be inserted into the wound while the cannula is retracted. In some embodiments, the device can be visualized as it is inserted by the applicator into the wound site. In some embodiments, the wound closure device seals the wound. Additionally, the wound closure device can facilitate wound in-growth. The wound closure device is adaptable to be inserted into the wound site without having to relocate the wound site opening.

Further provided herein is a wound closure device for use after ocular surgery comprising a plug adaptable to be inserted into an opening formed during ocular surgery, the opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening. The plug can be adaptable to transition between the first configuration and the second configuration after exposure to one or more of an aqueous medium, change in temperature, change of a physical environment, a pH, ion strength, salt concentration, change of a chemical environment, or light. In some embodiments, the plug comprises a biocompatible material. Additionally, the plug can be adaptable to be in the first configuration after being subjected to at least one of a physical force, a chemical force, and a mechanical force. The wound closure device can be adaptable to be inserted into the wound site without relocating the opening.

Further provided herein are methods for closing an opening following a vitrectomy comprising obtaining access through the conjunctiva and sclera; and inserting a wound closure device into the conjunctiva and sclera, wherein the opening is formed in two or more layers of tissue, one tissue layer transposable relative to a second tissue layer. The method allows the wound closure device to be inserted into the wound without having to unnecessarily damage the surrounding tissue. The method can further comprise the step of cutting the wound closure device. In some embodiments of the method, the method can further comprise the step of positioning the conjunctive over the wound closure device. The conjunctive can be actively positioned over the wound closure device by lifting the conjunctiva over the device. Alternatively, the conjunctiva can slide passively over the wound closure device. The method can provide for a wound closure device, where the wound closure device comprises a material having a first configuration and a second configuration, wherein the device is adaptable to be inserted into a wound in the first configuration and wherein the material transitions from the first configuration to the second configuration after being inserted into the wound. In some embodiments of the method, the wound closure device is adaptable to transition between the first configuration and the second configuration after being exposed to one or more of an aqueous medium, change in temperature, pH, ion strength, salt concentration, change of a chemical environment, change of a physical environment, or light, or any other suitable condition to which the material is exposed. In some embodiments of the method, after the access through the conjunctiva and sclera are obtained, a cannula or any suitable structure can be inserted though the access route. Furthermore, in some embodiments, the method can further comprise the step of removing the cannula from the access route after the wound closure device has been inserted through the cannula. In some embodiments of the method, the wound closure device remains fixed in position as the cannula is being removed. Alternatively, the wound closure device can be partially retracted while the cannula is being removed. The wound closure device can be retracted at the same time the cannula is removed. Alternatively, the wound closure device can be retracted after the cannula has been removed. Additionally, the method can provide for the step of the inserting a catheter through the cannula, wherein the catheter is adaptable to facilitate the insertion of the wound closure device. The catheter can be used to insert the wound closure device into the cannula before the cannula is removed. Alternatively, the catheter can be inserted into the cannula, the cannula removed, and then the wound closure device inserted into the opening. The cannula can be inserted into the opening by pushing, blowing, or moving the wound closure device by any suitable method for positioning the device in the opening. In some embodiments of the method, the method can provide for the step of inserting a guide wire through the cannula, wherein the guide wire is adaptable to facilitate the insertion of the wound closure device. The guide wire can be used to insert the wound closure device into the cannula before the cannula is removed. Alternatively, the guide wire can be inserted into the cannula, the cannula removed, and then the wound closure device inserted into the opening. The wound closure device can be located over the guide wire. Furthermore, in some embodiments of the method, the method can further comprise the step of severing the cannula, wherein a portion of the severed cannula is adaptable to facilitate closing the wound. In some embodiments, the cannula can be severed across the top, so that the external portion of the cannula is removed from the remainder of the wound closure device. The cannula can then be filled with a suitable wound closure device. Alternatively, the exterior of the cannula that comes in contact with the opening can have a sleeve of a biocompatible material. The interior of the cannula can be removed from the opening so that the sleeve remains within the opening. The interior of the sleeve remaining within the opening can then be filled with a suitable wound closure device. In some embodiments of the method, the wound closure device used is a non-solid material. In some embodiments of the method, the wound closure device is a solid material. The method can further comprise the step of delivering a drug to the vitreous chamber of the eye, wherein the drug is delivered by the wound closure device.

Another method provided herein is a method for closing a wound following a vitrectomy comprising obtaining access through a portion of a conjunctiva and a sclera through a cannula; and inserting a wound closure device through the cannula, wherein the access is an opening formed in two or more layers of tissue, one tissue layer transposable relative to a second layer. Furthermore, the method can provide for the step of cutting the wound closure device after the wound closure device has been positioned in the opening. In some embodiments of the method, the method can further comprise the step of positioning the conjunctiva over the wound closure device. The conjunctiva can be actively positioned over the wound closure device by lifting the conjunctiva over the device. Alternatively, the conjunctiva can slide passively over the wound closure device. The method can further provide for the use of a wound closure device comprising a material having a first configuration and a second configuration, wherein the device is adaptable to be inserted into a wound in the first configuration and wherein the material transitions from the first configuration to the second configuration after being inserted into the wound. The material can transition from a first configuration to a second configuration after being exposed to one or more of an aqueous medium, change in temperature, a chemical environment, pH, ion strength, salt concentration, or light. In some embodiments, the method can provide for the step of removing the cannula after the wound closure device has been inserted through the cannula. In some embodiments, the wound closure device remains stationary in the wound as the cannula is being removed from the wound. In some embodiments, the wound closure device can be partially retracted as the cannula is removed. The wound closure device can be inserted directly into the cannula. Alternatively, a catheter can be inserted into a cannula, and the cannula used to facilitate the insertion of the device into the opening. The device can be preloaded in the catheter. Alternatively, the catheter can be inserted into the cannula and then the device loaded in the catheter. The catheter can then introduce the device into the opening. In some embodiments, the catheter is inserted into the cannula and the cannula removed. The device can then be introduced into the opening after the cannula has been removed. The device can be pushed into the opening using a pusher rod extending through the catheter. Alternatively, the device can be drawn into the opening through capillary action. The device can be introduced into the opening using any suitable force for introducing the device into the opening. In some embodiments, the wound closure device can be introduced into an opening using a guide wire. The guide wire can be inserted into the cannula and the device introduced into the cannula using the guide wire. In some embodiments, the device is preloaded on the guide wire. In some embodiments, the guide wire is introduced into the cannula and then the device loaded on the guide wire. The guide wire can also be introduced into the cannula and then the cannula removed from the opening. The device can then be introduced to the opening using the guide wire. In some embodiments of the method, the method comprises the use of a cannula which can be used to close the wound. In such an embodiment a portion of the cannula can be used to close the wound. In some embodiments, the part of the cannula external to the eye can be severed. The remainder of the cannula can remain in the opening. The interior lumen of the cannula can then be filled with a wound closure device. Alternatively the exterior of the portion of the cannula post located within the wound can be severable from the top and interior part of the cannula post. As the cannula is withdrawn from the opening, the exterior portion of the post remains in the opening. The interior lumen of the coating can then be filled with a wound closure device. In some embodiments, the wound closure device comprises a non-solid material including, but not limited to, a gel, paste, or any other suitable non-solid material. In some embodiments, the wound closure device comprises a solid material including, but not limited to a polymer, or any other suitable biocompatible material.

Further provided herein is a method for closing an indexed wound using a wound closure device. An indexed wound comprises at least two layers of tissue, where one tissue has been transposed or displaced from its original position. The transposed tissue can be held in its displaced position during a procedure, thus being indexed. The method for closing an indexed wound using a wound closure device can comprise inserting a wound closure device through a wound without causing further trauma to the wound or an area surrounding the wound, the wound closure device having a first configuration and a second configuration, wherein the device is adaptable to be inserted into the wound in the first configuration and wherein the device is adaptable to transition to the second configuration after the device has be inserted into the wound.

Another embodiment of the method disclosed here is a method for closing a wound through which a procedure can be performed wherein the wound extends through at least two layers of tissue, the method comprising identifying a position of a wound; inserting a wound closure device into the wound; and closing the wound with the wound closure device, wherein the wound is formed in two or more layers of tissue, one tissue layer transposable relative to a second layer. In some embodiments, the wound is an ocular wound.

Also provided herein are kits comprising the invention disclosed herein. Provided herein is a kit for closing an opening following a vitrectomy procedure comprising a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening. The kit can further comprise at least one cannula. Additionally, the kit can comprise at least one catheter. The kit can also comprise at least one guide wire. In some embodiments, at least one catheter and one guide wire can be included in the kit. In some embodiments of the kit, the wound closure device can further comprise a drug eluting segment. The drug eluting segment can be preloaded with a drug. Alternatively, the drug eluting segment can be a loadable drug eluting segment, wherein a drug is loaded into the drug eluting segment. Furthermore, the kit provided herein can further comprise at least one vial comprising at least one drug. Multiple vials may be included with the kit. In some embodiments, the multiple vials contain the same drug. In some embodiments, the multiple vials contain different drugs. An amount of one kind of drug can be introduced to the drug eluting chamber. An amount of more than one kind of drug can be introduced to the drug eluting chamber to create a drug cocktail.

Another embodiment of a kit provided herein is a kit for closing a wound following a vitrectomy procedure comprising a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening; and a plug applicator adaptable to insert the plug. The kit can further comprise at least one cannula. Additionally, the kit can comprise at least one catheter. The kit can also comprise at least one guide wire. In some embodiments, at least one catheter and one guide wire can be included in the kit. In some embodiments of the kit, the wound closure device can further comprise a drug eluting segment. The drug eluting segment can be preloaded with a drug. Alternatively, the drug eluting segment can be a loadable drug eluting segment, wherein a drug is loaded into the drug eluting segment. Furthermore, the kit provided herein can further comprise at least one vial comprising at least one drug. Multiple vials may be included with the kit. In some embodiments, the multiple vials contain the same drug. In some embodiments, the multiple vials contain different drugs. An amount of one kind of drug can be introduced to the drug eluting chamber. An amount of more than one kind of drug can be introduced to the drug eluting chamber to create a drug cocktail.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a cross-sectional view of an eye;

FIG. 2A illustrates one embodiment of a wound closure device in a compressed state; FIG. 2B illustrates a cross-sectional view of the wound closure device shown in FIG. 2A along the line B-B; FIG. 2C illustrates one embodiment of a wound closure device in an expanded state; FIG. 2D illustrates a cross-sectional view of the wound closure device shown in FIG. 2C along the line B-B; FIG. 2E illustrates one embodiment of the wound closure device in use; FIG. 2F shows a cross-sectional view of the wound closure device in use under a tissue layer;

FIG. 3A illustrates a cross-sectional view of another embodiment of a wound closure device; FIG. 3B illustrates one embodiment of the wound closure device of FIG. 3A in a compressed form; FIG. 3C illustrates another embodiment of the wound closure device in a compressed form;

FIGS. 4A-4L illustrate alternate embodiments of the wound closure device;

FIG. 5 illustrates the device in use;

FIGS. 6A-6H illustrate the steps involved in using the device; FIG. 6A illustrates a cannula placed for a surgical procedure; FIG. 6B illustrates the tissue without the cannula in place; FIG. 6C illustrates a cannula placed for a surgical procedure; FIG. 6D illustrates the device being applied through the cannula; FIG. 6E illustrates the device being partially expanded to is second configuration; FIG. 6F illustrates the removal of the applicator and the cannula; FIG. 6G illustrates the device after it has been cut; FIG. 6H illustrates the device under a layer of tissue;

FIGS. 7A-7D illustrate one embodiment of the device wherein the device is deployed using a catheter;

FIGS. 8A-8D illustrate an alternate embodiment of the device wherein the device is deployed using a catheter;

FIGS. 9A-9D illustrate an embodiment of a wound closure device wherein a guide wire is used to deploy the device;

FIGS. 10A-10D illustrate an alternate embodiment of a wound closure device wherein a guide wire is used to deploy the device;

FIGS. 11A-11C illustrate an embodiment of a wound closure device wherein the wound closure device is a liquid;

FIGS. 12A and 12B illustrate an alternate embodiment of a wound closure device;

FIGS. 13A and 13B illustrate an alternate embodiment of a wound closure device;

FIG. 14A and 14B illustrate an alternative embodiment of a pressure fit wound closure device;

FIGS. 15A-15G illustrate alternate embodiments of a wound closure device comprising various embodiments of drug delivery units;

FIGS. 16A-16D illustrate a wound closure device and an alternate embodiment of a wound closure device applicator;

FIG. 17 shows an alternate embodiment of a wound closure comprising an additional feature; and

FIG. 18 is a graph illustrating the wound leakage rate per time for different wound site conditions in a rabbit eye.

FIG. 19A illustrates a drug eluting segment having one chamber; FIG. 19B illustrates a drug eluting segment having more than one chamber.

FIG. 20A illustrates a drug eluting segment comprising a micro-fluidic device; FIG. 20B is a longitudinal cross-section of the drug eluting segment shown in FIG. 20A; FIG. 20C illustrates a lateral cross-section of one embodiment of a micro-fluidic device; FIG. 20D illustrates a lateral cross-section of another embodiment of micro-fluidic device.

FIG. 21A illustrates a drug eluting chamber having a slit in the exterior surface from which a drug is eluted; FIG. 21B illustrates a drug eluting chamber incorporating a micro-fluidic device.

FIG. 22A illustrates a drug eluting segment with another embodiment of a micro-fluidic device; FIG. 22B illustrates a longitudinal cross section of the drug eluting segment shown in FIG. 22A; FIG. 22C illustrates a lateral cross section of the drug eluting segment shown in FIG. 22A; FIG. 22D illustrates the drug eluting segment shown in FIG. 22A eluting a drug.

FIG. 23A illustrates a drug eluting chamber having multiple compartments; FIG. 23B illustrates a drug eluting chamber having multiple micro-fluidic devices.

### DETAILED DESCRIPTION OF THE INVENTION

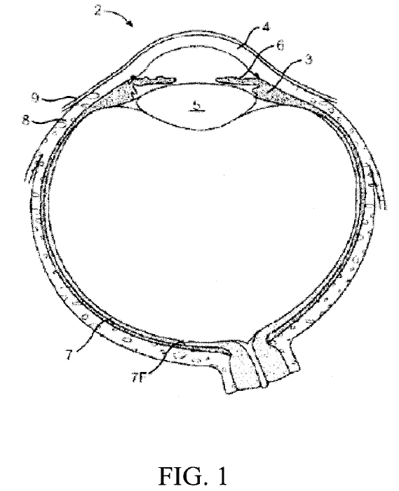

Provided herein are wound closure devices for closing a wound. The device can be used to close a wound in the eye. FIG. 1 is a representative illustration of the anatomical tissue structures of an eye 2. The eye 2 includes a cornea 4 and an iris 6. A sclera 8 is the white colored tissue that surrounds the cornea 4 and the iris 6. The conjunctival layer 9 is substantially transparent and is located over the sclera 8. A crystalline lens 5 is located within the eye. The retina 7 is located near the back of eye 2 and is generally sensitive to light. The retina 7 includes a fovea 7F that provides high visual acuity and color vision. The cornea 4 and lens 5 refract light to form an image on the fovea 7F and retina 7. The optical power of cornea 4 and lens 5 contribute to the formation of images on fovea 7F and the retina. The relative locations of cornea 4, lens 5 and fovea 7F are also important to image quality. For example, if the axial length of eye 2 from cornea 4 to retina 7F is large, the eye 2 can be myopic. Also, during accommodation, the lens 5 moves toward the cornea 4. This provides good near vision of objects proximal to the eye.

Provided herein is a wound closure device can comprise a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening. The wound closure device can be transition between a first configuration and the second configuration after exposure to one or more of an aqueous medium, change in temperature, a chemical environment, pH, ion strength, salt concentration, or light. The wound closure device can be a biocompatible material. The biocompatible material can be selected from at least one of a compressible material, temperature dependent material, shape memory material, a swellable material, and an expandable material. Additionally, the wound closure device can comprise an anchor adaptable to prevent removal of the wound closure device from a wound. The anchor can be a physical feature or alternatively the anchor can comprise the exterior surface of the device, where the exterior of the device undergoes a change in properties causing the device to anchor into the wound. The wound closure device can comprise a handle adaptable to insert the wound closure device in a wound. In some embodiments, the wound closure device is adaptable to be cut. Furthermore, the device can comprise markers along the length of the wound closure device to indicate depth of insertion of the device and to facilitate cutting of the device. Additionally, the wound closure device can comprise a drug delivery element. The wound closure device can be induced into the first configuration using at least one of a physical force, a chemical force, or a mechanical force. The wound closure device can be inserted into a wound using a device applicator where the device is a pre-cut device. Alternatively, the device can be cut by the applicator after being inserted into the wound. The wound closure device applicator can insert the wound closure device into the wound through a cannula. The device can be inserted into the wound while the cannula is retracted. In some embodiments, the device can be visualized as it is inserted by the applicator into the wound site. In some embodiments, the wound closure device seals the wound. Additionally, the wound closure device can facilitate wound in-growth. The wound closure device is adaptable to be inserted into the wound site without having to relocate the wound site opening.

Further provided herein is a wound closure device for use after ocular surgery comprising a plug adaptable to be inserted into an opening formed during ocular surgery, the opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening. The plug can be adaptable to transition between the first configuration and the second configuration after exposure to one or more of an aqueous medium, change in temperature, a change in the chemical environment, a change in the physical environment, pH, ion strength, salt concentration, or light. In some embodiments, the plug comprises a biocompatible material. Additionally, the plug can be adaptable to be in the first configuration after being subjected to at least one of a physical force, a chemical force, and a mechanical force. The wound closure device can be adaptable to be inserted into the wound site without relocating the opening.

### I. MATERIALS

The wound closure device with or without the drug delivery units can comprise one or more biocompatible materials. A non-biodegradable wound closure device can include silicone, acrylates, polyethylenes, polyurethane, polyurethane, hydrogel, polyester (e.g., DACRONB from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, polymer foam, silicone rubber, polyethylene terephthalate, ultra high molecular weight polyethylene, polycarbonate urethane, polyurethane, polyimides, stainless steel, nickel-titanium alloy (e.g., Nitinol), titanium, stainless steel, cobalt-chrome alloy (e.g., ELGILOYB from Elgin Specialty Metals, Elgin, Ill.; CONICHROMEB from Carpenter Metals Corp., Wyomissing, Pa.). A biodegradable wound closure device can comprise, one or more biodegradable polymers, such as protein, hydrogel, polyglycolic acid (PGA), polylactic acid (PLA), poly(Llactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof. In some embodiments the wound closure can comprise at least one of hydrogel polymer. The wound closure device can also comprise a combination of a non biodegradable and a biodegradable material. Further the wound closure can comprise two or more biodegradable material with different degradation durations.

### II. THERAPEUTIC AGENTS

The wound closure system can be used to deliver therapeutics agent to the wound site or to the surrounding tissue. Exemplary therapeutic agents include, but are not limited to, thrombin inhibitors; antithrombogenic agents; thrombolytic agents; fibrinolytic agents; vasospasm inhibitors; vasodilators; antihypertensive agents; antimicrobialagents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflaTnmatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non steroidal anti-inflammatories (NSAIDs) (such as salicylate, indomethacin, ibuprofen, diclofenac, flurbiprofen, piroxicam indomethacin, ibuprofen, naxopren, piroxicam and nabumetone). Such anti inflammatory steroids contemplated for use in the methodology of the present invention, include triamcinolone acetonide (generic name) and corticosteroids that include, for example, triamcinolone, dexamethasone, fluocinolone, cortisone, prednisolone, flumetholone, and derivatives thereof.); antiallergenics (such as sodium chromoglycate, antazoline, methapyriline, chlorphe-niramine, cetrizine, pyrilamine, prophenpyridamine); proliferative agents (such as 1,3-cis retinoic acid, 5-fluorou-racil, taxol, rapamycin, mitomycin C and cisplatin); decon-gestants (such-as ihenylephrine, naphazoline, tetrahydrazo-line); miotics and anti-cholinesterase (such as pilocarpine, salicylate, carbachol, acetylcholine chloride, physostigmine, eserine, diisopropyl fluorophosphate, phospholine iodinebromide); antineoplastics (such as carmustine, roidscisplatin, fluorouracil3; immunological drugs (such as vaccines and immune stimulants); hormonal agents (such as estrogens, -estradiol, progestational, progesterone, insulin, calcitonin, parathyroid hormone, peptide and vasopressin hypothalamus releasing factor); immunosuppressive agents, beta1 and beta2 (non-selective) adrenergic receptor blocking growth hormone antagonists, growth factors (such as epi-dermal growth factor, fibroblast growth factor, platelet derived growth factor, transforming growth factor betasomatotrapin, fibronectin); inhibitors of angiogenesis as angiostatin, anecortave acetate, thrombospondin, VEGF antibody); dopamine agonistsagents; peptides; proteins; enzymes; extracellular matrix;ACE inhibitors; freeradical scavengers; chela-tors; antioxidants; anti polymerases; photodynamic therapy agents; gene therapy agents; and other therapeutic agents such as prostaglandins, antiprostaglandins, prostaglandin, precursors, including antiglaucoma drugs including betablockers such as Timolol, betaxolol, levobunolol, atenolol, and prostaglandin analogues such as Bimatoprost, travoprost, Latanoprost etc; carbonic anhydrase inhibitors such as acetazolamide, dorzolamide, brinzolamide, methazolamide, dichlorphenamide, diamox; and neuroprotectants such as lubezole, nimodipine and related compounds; and parasympathomimetrics such as pilocarpine, carbachol, physostigmine and the like, or any suitable combination thereof.

### III. DEVICES

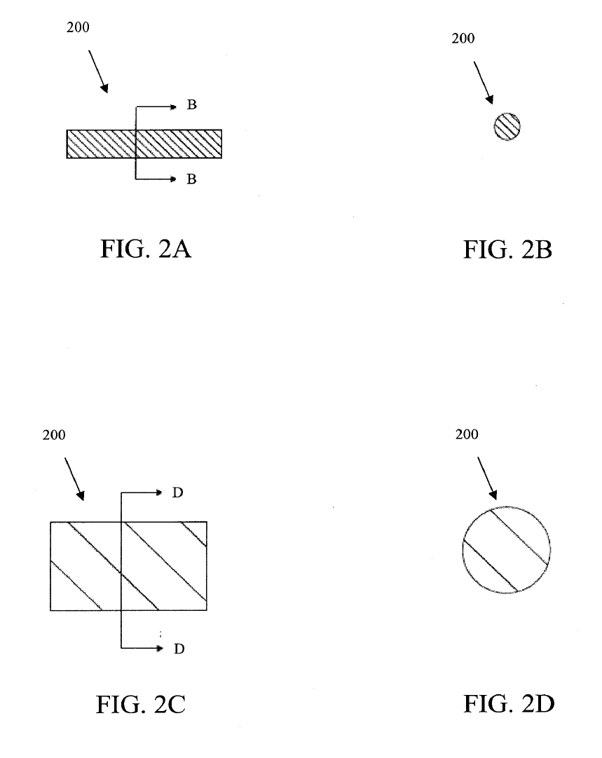

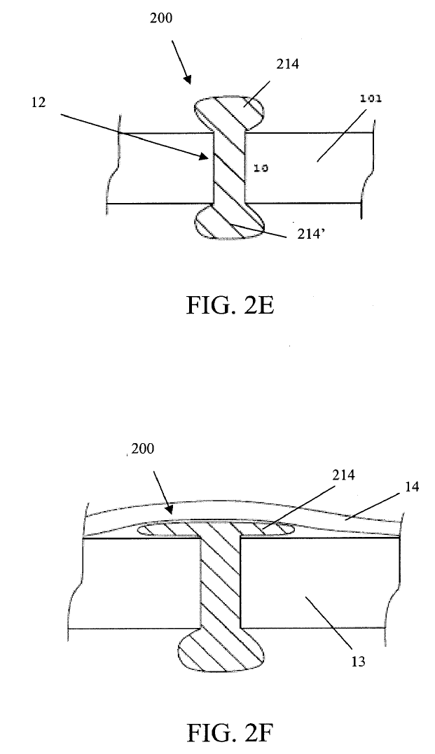

FIG. 2A is a side cross-sectional view of a wound closure device 200. The wound closure device 200 exists in a first configuration and a second configuration. FIG. 2A illustrates a wound closure device 200 in the first configuration before the device is inserted into the wound. FIG. 2B is a cross-sectional view of the wound closure device 200 shown in FIG. 2A along the line B-B. FIG. 2C illustrates a wound closure device 200 in the second configuration. A cross-sectional view of the device 200 in FIG. 2C along the line D-D is shown in FIG. 2D. The cross-sectional area can be circular, as shown in FIG. 2C. Alternatively, the cross-sectional area can have any suitable cross-sectional area including, but not limited to, square, rectangular, polygonal, or an amorphous cross-sectional area. In some embodiments the volume of the wound closure device in the first configuration is smaller the volume of device in the second configuration. In other embodiments, the diameter of the wound closure device in the first configuration is smaller than the diameter of the device in the second configuration. In still other embodiments, both the volume and diameter of the device in the first configuration is smaller than the volume and diameter of the device in the second configuration. In the first configuration, the wound closure can be placed in the wound site with minimal or no manipulation of the tissue located close to the wound site. The wound closure device can transition from the first configuration to the second configuration due to external environmental cues including, but not limited to, thermal, physical, or chemical envirormental cues.

FIG. 2E illustrates a wound closure device 200 in use wherein the wound closure device 200 is being used to close a wound 12 in a tissue layer 10. After transitioning to the second configuration, the wound closure device 200 can comprise anchor units 214, 214′ located on both sides of the tissue layer 10. In some embodiments, one anchor 214 can be located on one side of the tissue layer 12 and a second anchor 214′ can be located on the other side of the tissue layer 12. In some embodiments, the wound closure device can comprise a material that partially seals the wound. Alternatively, the wound closure device can comprise a material that completely seals the wound. The wound closure device can be comprised of a biocompatible material. In some embodiments, the wound closure device is a biodegradable material. As the biodegradable material degrades, tissue in-growth can reconstruct the wound site.

FIG. 2F illustrates a cross-sectional side view of the wound closure device 200 in its second configuration employed in a wound site 12 of a first tissue layer 13 and located underneath a second tissue layer 14. An anchor unit 214 is located between the bottom and the top layer. The anchor unit 214 undergoes a change in response to the top tissue layer 214 and forms a flat shield structure which stabilizes, mechanically, the wound closure device 200. The flattening of the anchor unit 214 causes minimal deformation of the second tissue layer 14.

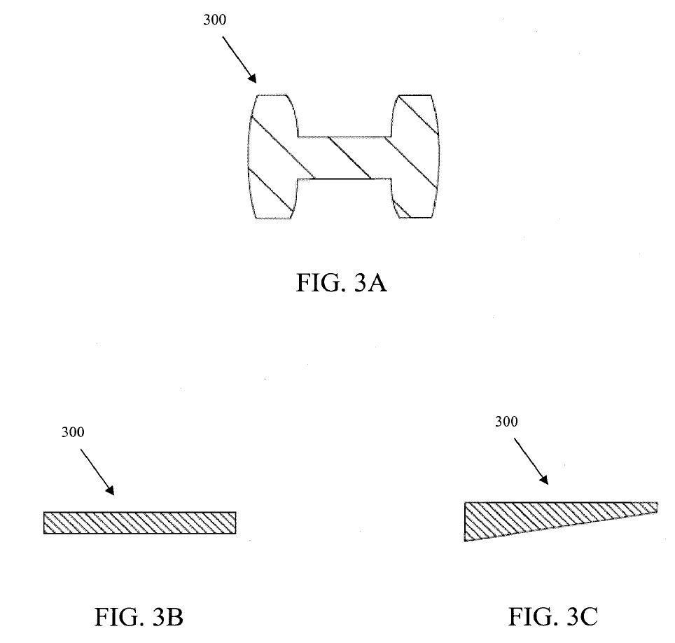

FIG. 3A illustrates a cross-sectional side view of an alternative embodiment of the wound closure device 300 in its second expanded configuration. FIG. 3B illustrates one embodiment of the wound closure device 300 in its compressed configuration where the device 300 is uniformly compressed. FIG. 3C illustrates an alternate compressed embodiment of the wound closure device 300 where the device 300 is asymmetrically compressed

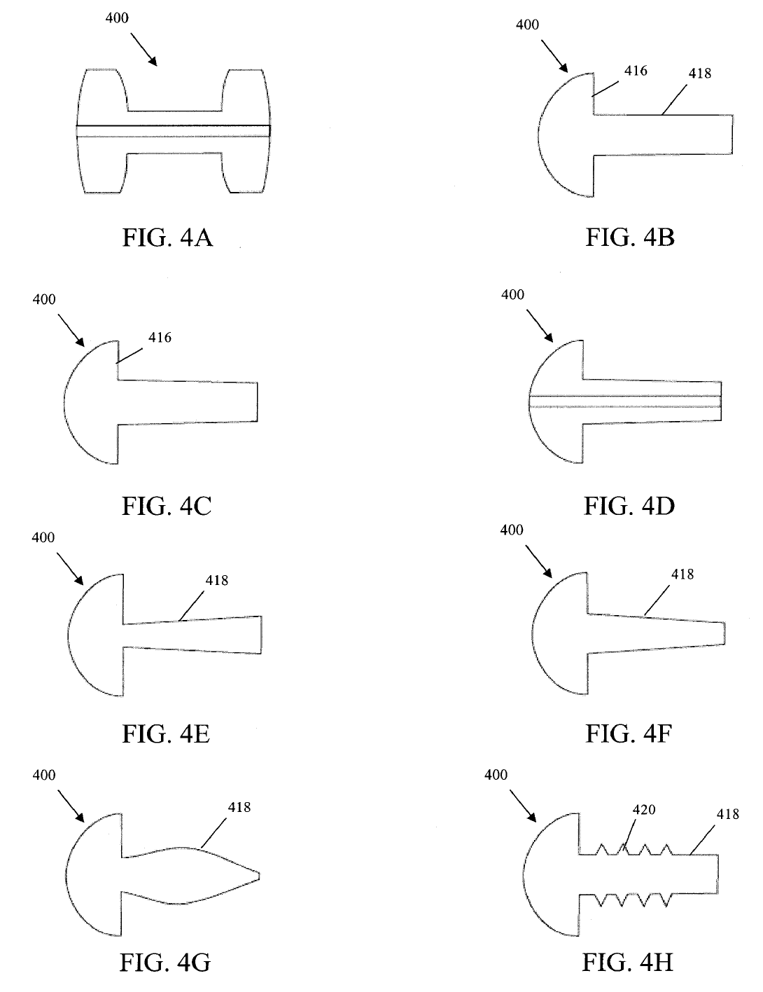

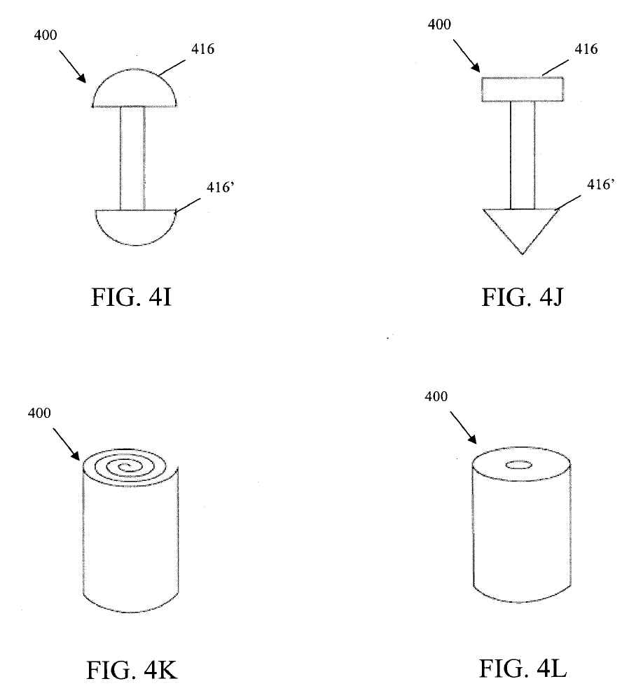

Alternative embodiments of the wound closure device are shown in FIGS. 4A-4L. The wound closure device can be solid. Alternatively, the wound closure device can be hollow as shown in FIG. 4A. In some embodiments, the wound closure device 400 comprises a cap 416 at one end, as shown in FIG. 4B. The device with a cap can be solid as shown in FIG. 4B or the device can be hollow as shown in FIG. 4C. The device can have cap 416 and post 418 wherein the post 418 is of uniform diameter. Alternatively, the diameter of the post 418 can vary along the length of the post as shown in FIGS. 4E-4G. The post 418 can also comprise anchoring features 420 as shown in FIG. 4H. In some embodiments, the wound closure device has a cap at one end of the device. Alternatively, the wound closure device can have a cap 416, 416′ at both ends of the wound closure device 400, as shown in FIG. 4I. The caps 416, 416′ can be uniform at both ends as shown in FIG. 4I. Alternatively, the caps 416, 416′ can be different with respect to each other, as shown in FIG. 4J. Alternate embodiments of the wound closure device 400 are shown in FIGS. 4K and 4L.

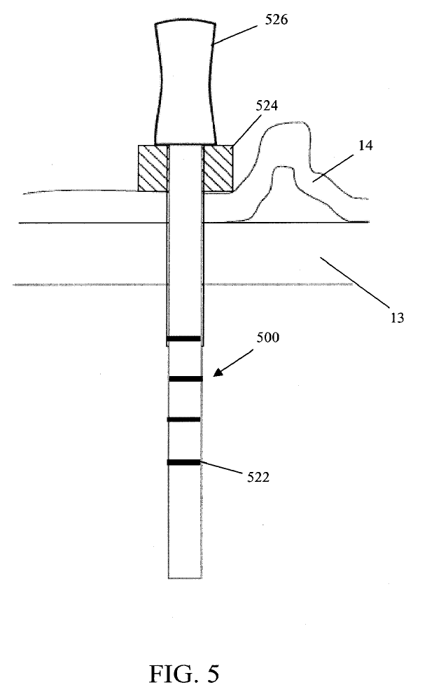

FIG. 5 illustrates one embodiment of a wound closure device 500 being used. The device can be used with a cannula 524 already positioned through a first 13 and second 14 layer of tissue during a surgical procedure. The device 500 can comprise a handle 526 to facilitate the insertion of the device 500. In some embodiments, the device can comprise markings 522 along the device 500 to indicate length for cutting.

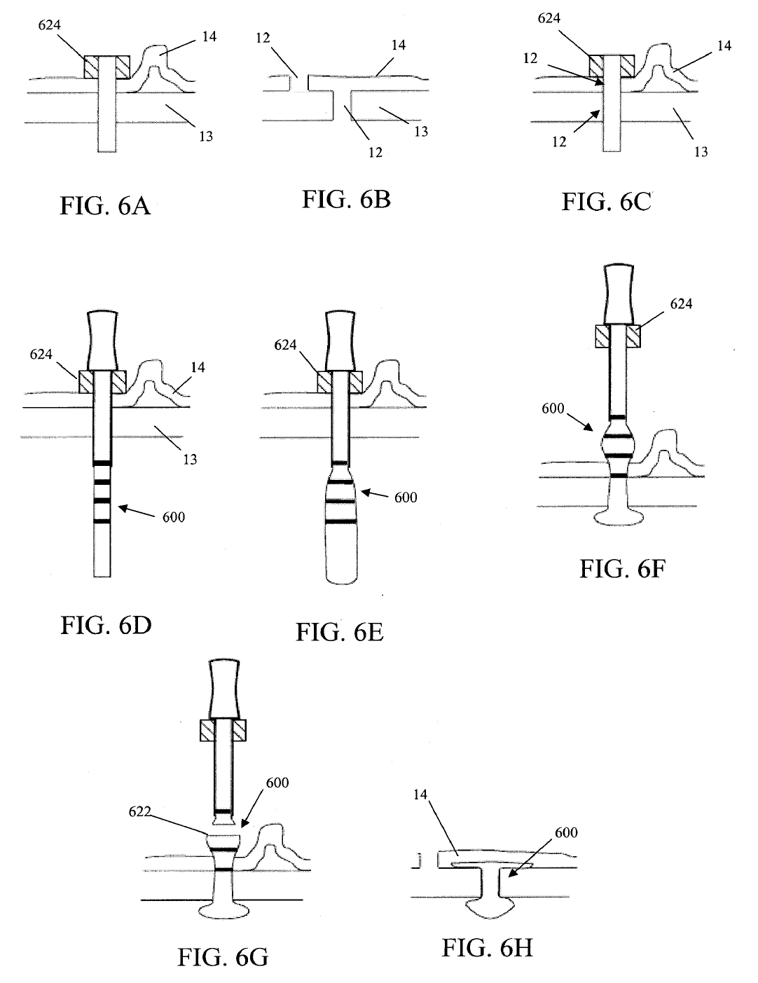

FIGS. 6A-6H illustrates the steps for using the device. FIG. 6A illustrates a cross section view of a placed cannula 624 in tissue layers 13, 14. The cannula can be placed during a sutureless vitrectomy procedure. The second layer 14 is shifted from its resting position during the placement of the cannula 624 by using a trocar. After removal of the cannula the second layer 14 slides back to its original location, as shown in FIG. 6B. The shifting of the second layer covers the wound site 12 of the first layer 13. In this case, access to the wound site 12 in the first layer cannot be gained without manipulation of the second layer. In the case of a vitrectomy procedure, the second layer 14 is represented by the conjunctiva and the top layer 13 is represented by the sclera of an eye.

The device can be inserted into the wound site using the cannula 624 positioned through the first 13 and second layers 14 of tissue. The cannula 624 can be used as an index tool to align the wound site 12 of the first layer 13 with the wound site in the second layer 14, as shown in FIG. 6C. The wound closure device 600 can be inserted into the cannula 624, as shown in FIG. 6D. After the wound closure device 600 is placed into the cannula 624, the protruding part of the wound closure device 600 changes from the first configuration to the second configuration as shown in FIG. 6E. After the wound closure device begins the transition from the first configuration to the second configuration, the cannula 624 is retracted. The wound closure device 600 can be partially retracted with the cannula 624 to the desired marking 622 as shown in FIG. 6F. The wound closure device 600 can then be cut to the desired length using the markings 622 as an indicator, as shown in FIG. 6G. The second tissue layer 14 can then be slid over the wound closure device 600 as shown in FIG. 6H.

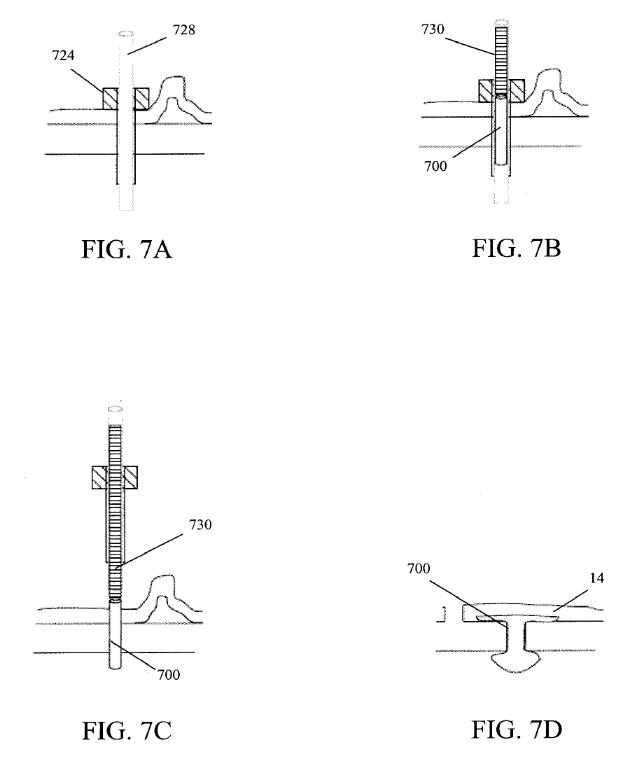

In some embodiments, the device can be inserted into the wound using a cannula. In some embodiments, the wound closure device can be positioned into an indexed wound wherein the indexed wound remains indexed due to any suitable means for maintaining the indexed wound including, but not limited to, sutures or adhesives. In some embodiments, the wound closure device can be directly inserted into a wound through a cannula. In some embodiments, the wound closure device can be inserted into the cannula using a catheter or tube 728 as shown in FIG. 7A. The tube 728 can be preloaded with a wound closure device 700. Alternative, the wound closure device 700 can be inserted into the tube 728 after the tube 728 has been inserted into the cannula 724 as shown in FIG. 7A and FIG. 7B. The wound closure device 700 can be precut and placed into the tube 728 in its first configuration. Alternatively, the wound closure device can be positioned in the wound using the tube 728 and then cut to the desired length. In some embodiments, the wound closure device 700 can be inserted into the wound site using a plunger 730. The cannula 724 and the catheter 728 can then be removed from the wound site 12 leaving the wound closure device 700 in position in the wound site 12, as shown in FIG. 7C. After the wound closure device 700 is exposed to the environment, it transforms from its first configuration to its second configuration. FIG. 7D shows the wound closure device 700 in its second configuration underneath a layer of tissue 14.

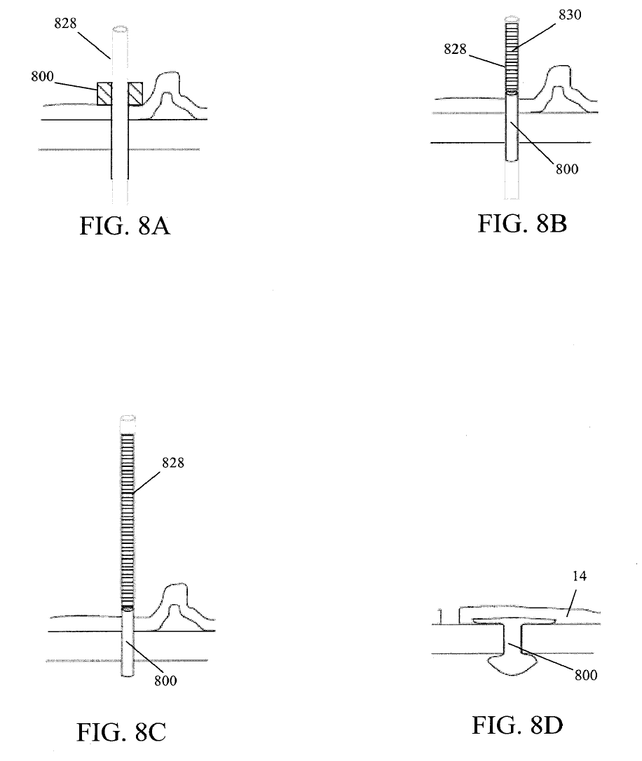

An alternate embodiment of a wound closure device 800 wherein the wound closure device 800 is positioned within the wound site 12 using a cannula is shown in FIGS. 8A-8D. FIG. 8A illustrates the placement of the catheter 828 within the cannula 824. The cannula 824 can then be removed from the wound site, leaving only the catheter 828 in the wound site. The device 800 can then be introduced to the wound site through the catheter 828 as shown in FIG. 8B. In some embodiments, a plunger 830 can be used to position the device 800 within the wound site. Once the device 800 has been positioned within the wound site 12, the catheter 828 can then be removed leaving the device 800 in place, as shown in FIG. 8C. FIG. 8D shows the wound closure device 800 in its second configuration underneath a layer of tissue 14.

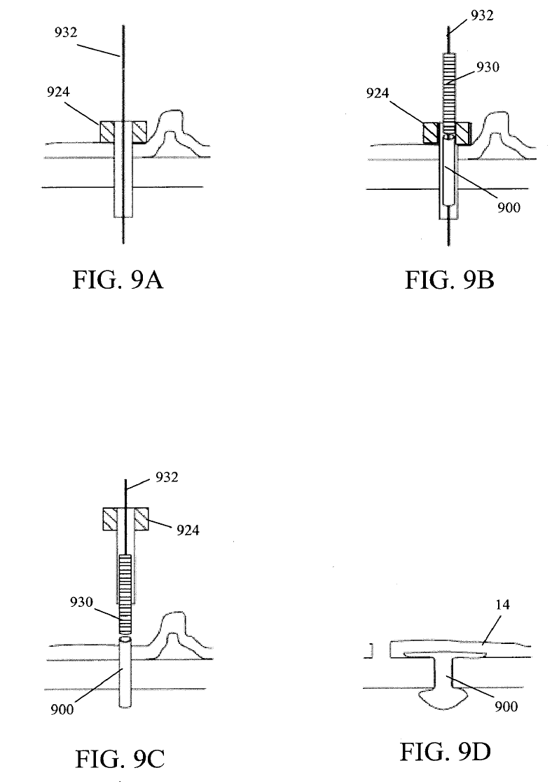

In some embodiments, the wound closure device 900 can be positioned within the wound site 12 using a guide wire 932 as shown in FIG. 9A. A guide wire 932 can be positioned within the cannula 924. The wound closure device 900 can be inserted into the wound site 900 using the guide wire as a guide, as shown in FIG. 9B. In some embodiments, the insertion of the wound closure device 900 can be facilitated using a plunger 930 as shown in FIG. 9B. In some embodiments the wound closure device can be precut. In some embodiments, the wound closure device can be cut after being positioned in the wound site. Once the device 900 has been positioned in the wound site 12, the cannula 924 and the guide wire 932 and plunger 930 can be removed, leaving the wound closure device 900 in place, as shown in FIG. 9C. After exposure to the external environment, the wound closure device 900 transitions from its first configuration to its second configuration. FIG. 9D shows the wound closure in its second configuration underneath a layer of tissue 14.

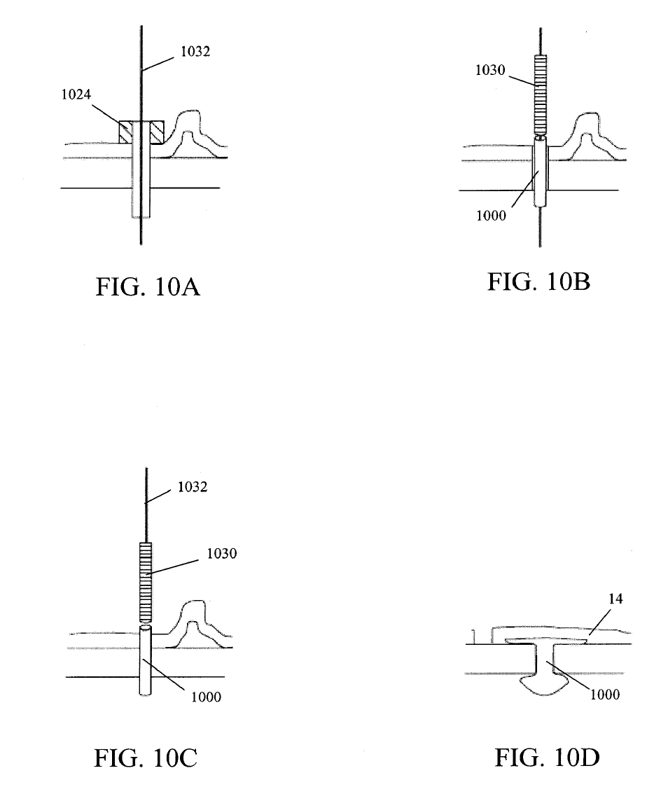

An alternate embodiment of a wound closure device 1000 wherein the wound closure device 1000 is positioned within the wound site 12 using a guide wire is shown in FIGS. 10A-10D. FIG. 10A illustrates the placement of the guide wire 1032 within the cannula 1024. The cannula 1024 can then be removed from the wound site, leaving only the guide wire 1032 in the wound site 12. The device 1000 can then be introduced to the wound site using the guide wire 1032 as shown in FIG. 10B. In some embodiments, a plunger 1030 can be used to position the device 1000 within the wound site. Once the device 1000 has been positioned within the wound site 12, the guide wire 1032 can then be removed leaving the device 1000 in place in the wound site 12, as shown in FIG. 10C. FIG. 10D shows the wound closure device 1000 in its second configuration underneath a layer of tissue 14.

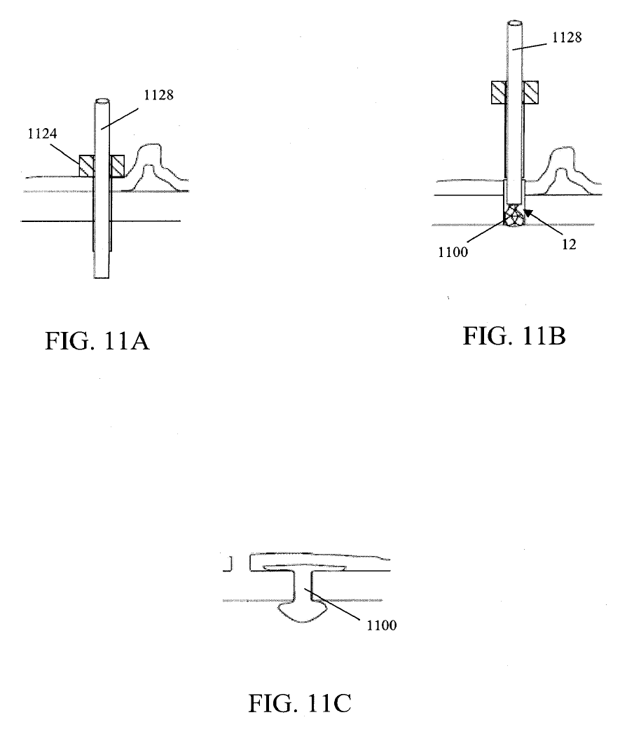

The wound closure device can be a solid structure in the first configuration. Alternatively, the wound closure device can be a liquid in the first configuration. FIGS. 11A-11C illustrates how a liquid wound closure device can be applied. A tube or catheter 1128 can be inserted in the cannula as shown in FIG. 11A. The wound closure device 1100 in its liquid configuration can then be dispensed at the wound site 12, as shown in FIG. 11B. After the liquid wound closure device 1100 is exposed to the wound site 12, it solidifies. Instant solidification can be achieved based on mechanisms such as, for example purposes only, cross linkage, polymerization, or phase transition triggered by the chemical or physical environment of the tissue layer. As shown in FIG. 11B, the wound closure can be dispensed out of the end of the tube 1128. In some embodiments, a liquid wound closure device can be applied to the wound site through the walls of the delivery tube. The liquid wound closure device can be delivered to the wound site using a spray head. A liquid wound closure device can be delivered to the wound site by any suitable method for delivering the wound closure device. In some embodiments, the wound closure device can be applied together with a carrier substance such as, for example purposes only, a gas or liquid. FIG. 11C illustrates the liquid wound closure device in its second configuration.

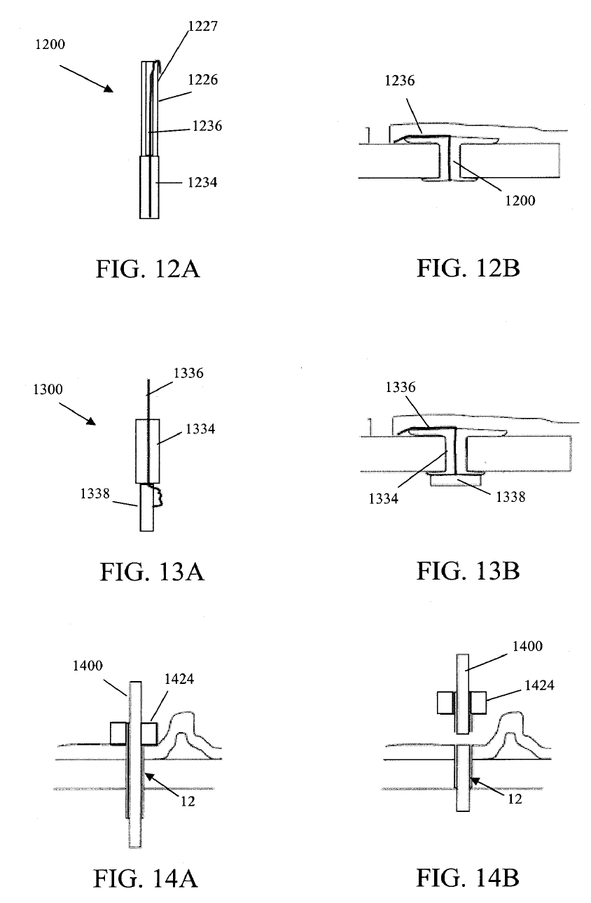

In some embodiments, the wound closure device 1200 comprises a sealing unit 1234, a handle 1226, and a connector 1236 for connecting the sealing unit 1234 to the handle 1226, as shown in FIG. 12A. The connector 1236 can be any suitable connector for connecting the handle to the sealing unit including, but not limited to, a string or wire. The connector can be connected to the handle by clamping the connector to the handle. Alternatively, the connector can be connected to the handle by adhering the connector to the handle using an adhesive. The connector can be connected to the handle by any suitable method for adhering the connector to the handle. The connector 1236 can be attached to the outside surface 1227 of the handle 1226, as shown in FIG. 12A, in order to keep the handle 1226 and the sealing unit 1234 together as a unit. The handle 1226 can be used to facilitate the insertion of the device into the cannula. After the sealing unit 1234 of the wound closure device 1200 is positioned, the sealing unit 1234 can be released from the handle 1226 by releasing the connector 1236 from the outside surface 1227 of the handle 1226. FIG. 12B illustrates a wound closure device 1200 in its second configuration.

An alternate embodiment of a wound closure device is shown in FIGS. 13A and 13B. A wound closure device 1300 can comprise a sealing unit 1334, an anchor unit 1338, and a connector 1336 for connecting the anchor unit 1338 to the sealing unit 1334, as shown in FIG. 13A. After the wound closure device 1300 is introduced to the wound site, the anchor unit 1338 can be manipulated using the connector 1336. In some embodiments, pulling on the connector can cause the anchor unit to rotate, thereby anchoring the sealing unit 1334. The sealing unit 1334 can then change from a first to a second configuration, as shown in FIG. 13B.

Yet another embodiment of a wound closure device 1400 is shown in FIGS. 14A and 14B. FIG. 14A shows a side cross-sectional view of a wound closure device 1400 positioned in a wound site 12. The wound closure device 1400 can comprise a solid rod in its first configuration. In some embodiments, the top part of the cannula 1424 and a portion of the inserted rod wound closure device 1400 can be removed by cutting the cannula 1424 and the rod 1400, as shown in FIG. 14B. Cutting of the cannula and the rod wound closure device can then cause the wound site to close. In some embodiments, the wound closure device can seal the wound site.

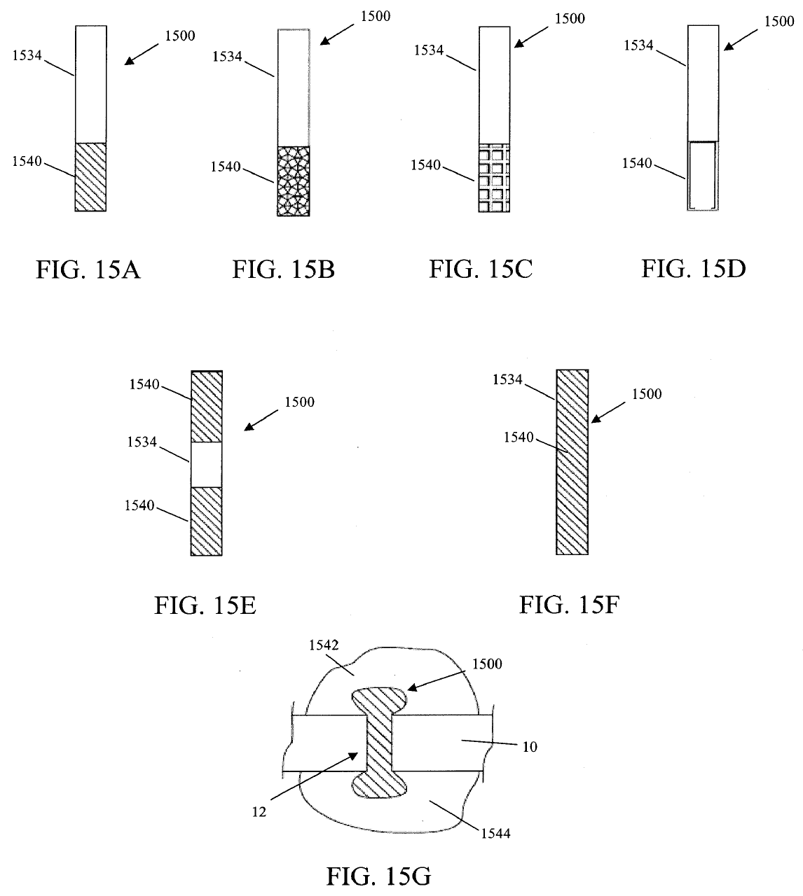

The wound closure device can serve to close a wound site. The wound closure device can also serve to close the wound closure device and release a therapeutic agent to the wound site. A wound closure device comprising a drug eluting segment is shown in FIGS. 15A-15G. FIG. 15A illustrates a wound closure device 1500 comprising sealing unit 1534 and a drug eluting segment 1540. The drug eluting segment can be attached directly to the sealing unit. Alternatively, a connector can be used to connect the drug eluting segment to the sealing unit. In some embodiments, the drug eluting segment 1534 is a solid structure, as shown in FIG. 15A. Alternative embodiments of drug eluting segments 1534 are illustrated in FIGS. 15B-15D. The drug eluting segment 1534 can be a porous matrix, as shown in FIG. 15B. Alternatively, the drug eluting segment 1534 can be comprised of a micro- or nanofluidic system, as shown in FIG. 15C. In yet another embodiment, the drug eluting segment 1534 can be a hollow structure that can be filled with a drug, as shown in FIG. 15D. In some embodiments, the drug eluting segment can be a single type of drug eluting segment. In some embodiments, the drug eluting segment can be a combination of drug eluting segment types. The drug eluting segment can be a biodegradable structure. The drug eluting segment can be any suitable structure for delivering a drug to the wound site. The drug eluting segment can be located at one end of the wound closure device 1500. Alternatively, a drug eluting segment 1540, 1540′ can be located on both ends of the wound closure device 1500, as shown in FIG. 15E. In a further embodiment of the drug eluting segment, the drug eluting segment 1540 can be located within the entire length of the wound closure device 1500, as shown in FIG. 15F. FIG. 15G illustrates yet another embodiment of a wound closure device 1500 being used to close a wound site 12. The wound closure device 1500 can be a porous structure or have channels that run longitudinally through the wound closure device 1500. The pores or channel size of the wound closure device 1500 can be sufficiently large to allow the passage of drugs. Preferably, the size of the channels and pores should be smaller than 1 micrometer. One end of a wound closure device 1500 can be connected to a drug depot 1542 on one side the tissue layer 10. The wound closure device 1500 can transports the drugs through the wound site 12 to the space 1544 on the other side of the tissue layer 10. In the case of a vitrectomy procedure, the top side of the tissue layer 10 represents the subconjunctival space and the bottom side of the tissue layer 10 represents the vitreous cavity. The wound closure device can enable the transport of drugs from subconjunctival space to the wound site or into the vitreous cavity.

The drug eluting segment can be used to deliver a drug to the wound site or to the interior space of the wound site. The drug eluting segment can be used to deliver a therapeutic agent to the wound site including, but not limited to, growth factors. Additionally, the drug eluting segment can be used to deliver saline to the wound site.

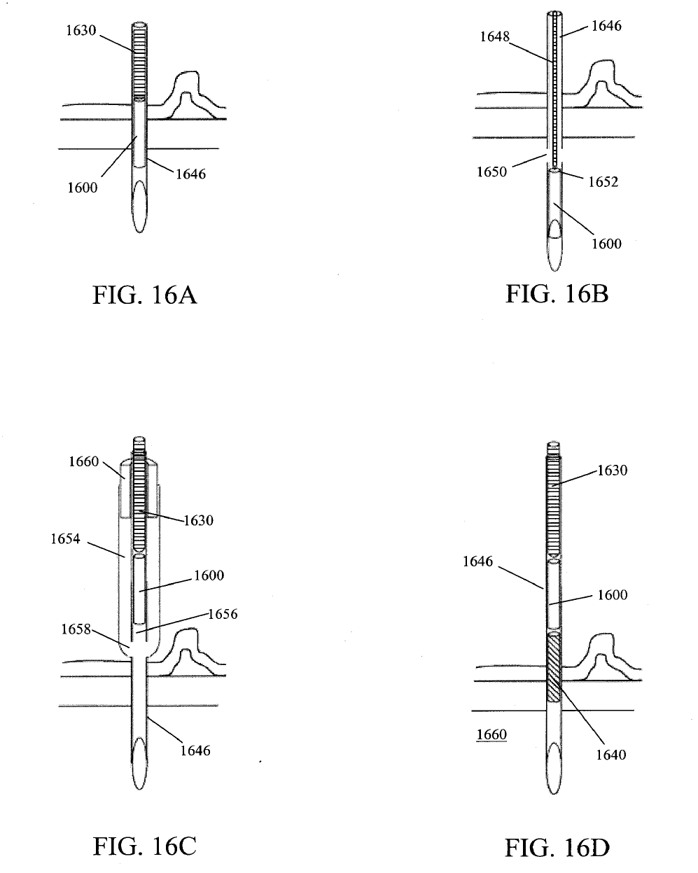

A wound closure system 1601 is shown in FIGS. 16A-16D. FIG. 16A illustrates a wound closure device 1600 located within an injection needle 1646. The wound closure device 1600 can be inserted into the wound site after injection of fluid into the space under the tissue layer using the needle 1646. Alternatively, the wound closure device 1600 can be inserted into the wound site after the withdrawal of fluid from the space underneath the tissue layer. The wound closure device 1600 can be positioned in the wound site using a plunger 1630, as shown in FIG. 16A. The wound closure system can be directly introduced through the layers of tissue. Alternatively, the wound closure system can be introduced through a cannula.

An alternative embodiment of a wound closure system 1601 for inserting a wound closure device 1600 into a wound site with an injection needle 1646 is shown in FIG. 16B. In FIG. 16B, the wound closure device is connected to a push rod 1648. In some embodiments, the push rod 1648 has a smaller diameter than the wound closure device 1600. The needle 1646 can have at least one opening 1650 in the wall 1652 of the needle 1646. The opening can be located above the position of the wound closure device 1600. Fluid can be injected into or extracted from the tissue without having to pass through the wound closure device 1600. After fluid has been injected into or retracted from the space underneath the tissue layer, the wound closure device 1600 can be positioned into the wound site. Alternatively, the wound closure system 1601 can be a closed system, wherein the fluid can be injected or retracted without an open wound site, as shown in FIG. 16C. The fluid can flow from a fluid chamber (tube) 1654 surrounding the in injection tube 1656. The wound closure device 1600, in its first configuration, is kept in the upper part of the injection tube 1656. The fluid chamber 1654 is connected to the injection tube 1607 through aperatures 1658 at the end of the fluid tube 1654. The side apertures 1658 are located below the position of the wound closure device 1600 in the injection tube 1656. Fluid can be injected into or retracted from the space underneath the tissue layers using a fluid plunger 1660 in, preferably, fluid communication with the fluid chamber 1654. During retraction of the injection needle 1646, the wound closure device 1600 can be placed with the plunger 1630 into the wound side of one or more tissue layers.

Another embodiment of a wound closure system 1601 using a needle 1646 is shown in FIG. 16D. The wound closure system 1601 can be comprised of a push rod and a drug delivery unit 1640. After delivering the drug delivery unit 1640 to the space 1660 underneath the tissue layers, the wound closure device 1600 can be deployed into the wound site without having an open wound site. In some embodiments, the wound delivery device can be delivered using a plunger 1630. Preferably, this system can be used to insert a sustained drug delivery device in the vitreous space.

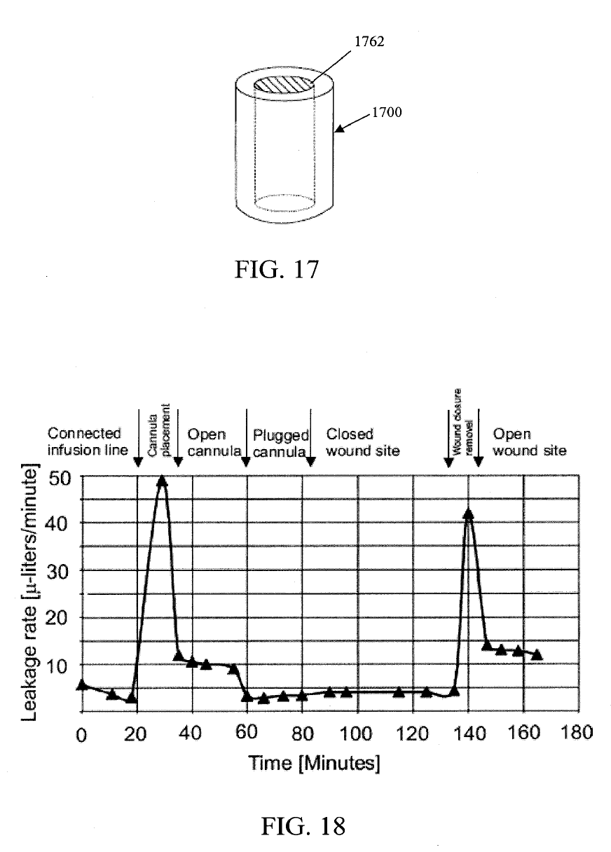

The wound closure system can be combined with an additional device feature, as conceptually shown in FIG. 17. The wound closure device 1700 can include an additional feature 1762 including, but not limited to, valves, sensors, actuators, or electronic circuits. In some embodiments, ports for injections or sampling can be embedded in the wound closure system. Any suitable additional feature can be used with the wound closure device. The implantation of the wound closure device 1700 can embed the additional feature into the tissue of the body or the eye.

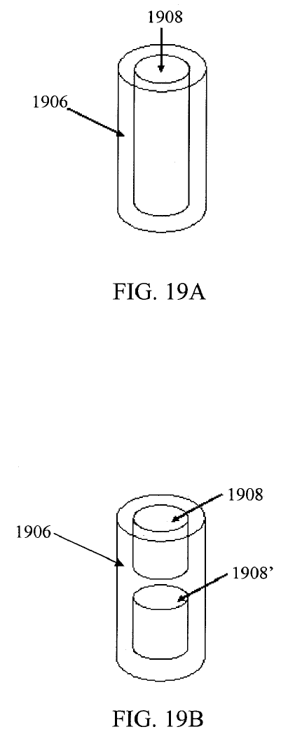

The wound closure device described herein, in addition to the wound plug and sealing segment, can comprise a drug eluting segment. An isolated drug eluting segment 1906 is shown in FIGS. 19A and 19B. FIG. 19A shows a drug eluting segment 1906 comprising a drug eluting chamber 1908. In some embodiments, the drug eluting chamber 1908 is a single chamber in which a single therapeutic agent is stored. The drug eluting chamber can be sized to contain the amount of therapeutic agent required. In some embodiments, the drug eluting segment 1906 can comprise more than one chamber, as shown in FIG. 19B. In FIG. 19B, a drug eluting segment 1906 is shown in which the drug eluting segment 1906 has two drug eluting chambers 1908, 1908′. The drug eluting chambers can contain the same therapeutic agent. The therapeutic agent can be released from the two chambers at different rates. In some embodiments, the therapeutic agents can be different therapeutic agents. The drug eluting segment can further comprise a hollow drug eluting chamber. Alternatively, the drug eluting segment can be solid. The drug eluting segment can comprise a biodegradable, bioresorbable, or bioabsorbable matrix that incorporates a therapeutic agent. As the matrix breaks down, the therapeutic agent can be released.

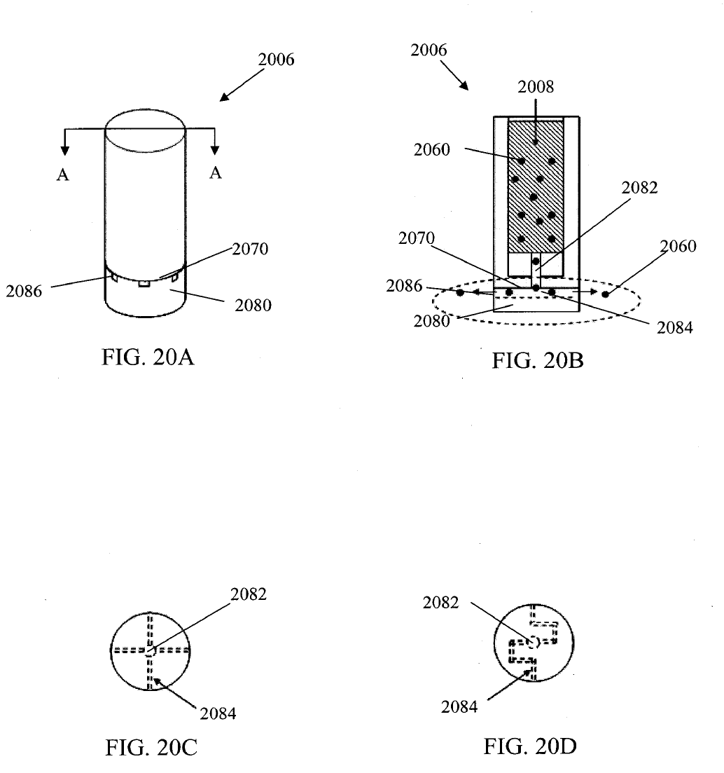

Different embodiments of the drug eluting segment can be used with the wound plug. The different embodiments can provide different mechanisms by which the therapeutic agent is released from the drug eluting segment. Different mechanisms can be used to control the rate at which a therapeutic agent is released from the drug eluting segment. FIGS. 20A-20C illustrate an embodiment of a drug eluting segment in which a micro-fluidic device is incorporated into the drug eluting segment to control the rate of release of the therapeutic agent. FIG. 20A is an external view of a drug eluting segment 2006 with a micro-fluidic device 2080 located at the distal end 2070 of the drug eluting segment 2006. A therapeutic agent can pass out of the device though a series of ports 2086 along the micro-fluidic device 2080. FIG. 20B is a cross section of the drug eluting segment 2006 shown in FIG. 20A along the line A-A. In FIG. 20B, the cross section of the drug eluting segment 2006 further illustrates a drug eluting chamber 2008 containing a therapeutic agent 2060. The micro-fluidic device 2080 located at the distal end 2070 of the drug eluting chamber 2006 has a connector 2082 providing communication between the drug eluting chamber 2008 and the micro-fluidic device 2080. Once the therapeutic agent 2060 passes from the drug eluting chamber 2008 to the micro-fluidic device 2080 through the connector 2082, the therapeutic agent 2060 flows through the micro-channels 2084 of the micro-fluidic device 2080 out the ports 2086. FIG. 20C illustrates one embodiment of a drug eluting segment including a micro-fluidic device as viewed from the end. FIG. 20C illustrates one design of micro-channels 2084 connected to the connector 2082. The orientation of the micro-channels controls the rate at which the therapeutic agent is released. Another embodiment of a device is illustrated in FIG. 20D. FIG. 20D illustrates a device comprising a micro-fluidic structure having a more convoluted micro-channel 2084 design connected to the connector 2082. A convoluted micro-channel can serve to provide a longer path for the therapeutic agent, thereby increasing the amount of time over which the therapeutic agent takes to reach the external environment. In some embodiments, only one micro-channel design is used with a micro-fluidic device. In some embodiments, more than one micro-channel design is used together in the same micro-fluidic device. In some embodiments, the micro-fluidic device has micro-channels that are symmetrical within the micro-fluidic device, each micro-channel being of the same design and spaced evenly apart with respect to each other. In some embodiments, the micro-channels vary throughout the micro-fluidic device and are unevenly spaced with respect to each other.

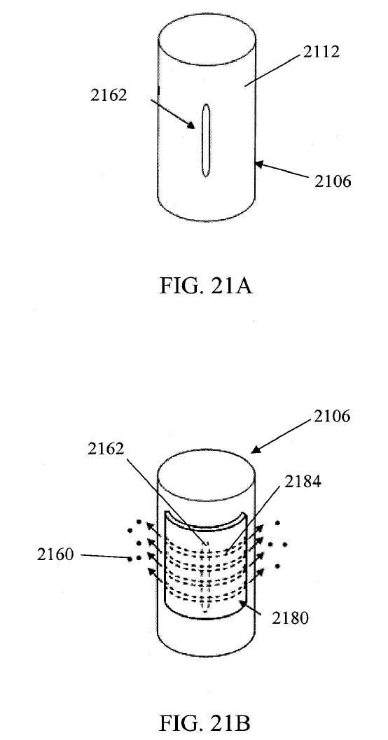

Another embodiment of a drug eluting segment is one having a micro-fluidic device patch for controlling the rate of release of a therapeutic agent as shown in FIGS. 21A and 21B. In such an embodiment, as shown in FIG. 21A, the drug eluting segment 2106 has a slit 2162 located in the exterior surface 2112 of the drug eluting segment 2106. The drug eluting segment 2106 is used as shown in FIG. 21A, without the further addition of a micro-fluidic device. Alternatively, a micro-fluidic device patch 2180 can be placed over the slit 2162 in the drug eluting segment 2106, as shown in FIG. 21B. In some embodiments, the micro-fluidic device patch 2180 is in fluid communication with the slit 2162. At least one micro-channel 2184 located within the micro-fluidic device patch 2180 is in communication with the slit 2162. In some embodiments, more than one micro-fluidic channel is located within the micro-fluidic device patch. A therapeutic agent 2160 can then travel through the micro-fluidic channel 2184 to the exterior space where the therapeutic agent 2160 then comes in contact with the wound.

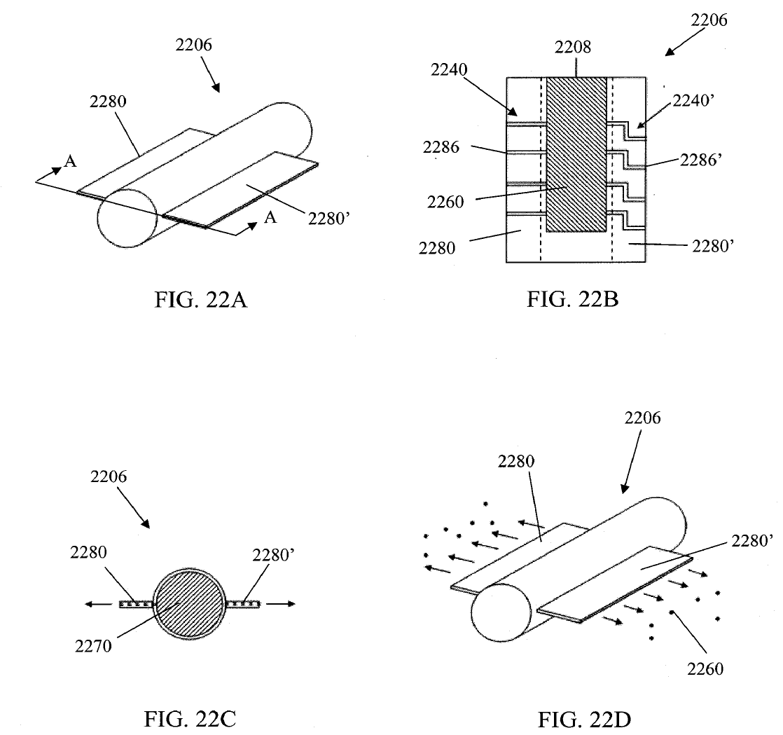

Another embodiment of a drug eluting segment is shown in FIGS. 22A-22D. FIG. 22A shows a perspective view of a drug eluting segment 2206 with a micro-fluidic device 2280, 2280′ extending from the segment. FIG. 22B is a cross section of the drug eluting segment 2206 shown in FIG. 22A along the line A-A. The therapeutic agent 2260 located in the drug eluting chamber 2208 can be in communication with the exterior space through at least one micro-fluidic channel 2284. In some embodiments, the therapeutic agent is in fluid communication with the exterior space. In some embodiments, more than one micro-fluidic channel 2284, 2284′, provides a passageway from the drug eluting chamber 2208 to the exterior space. In some embodiments, only one micro-fluidic design is used per device. More than one micro-fluidic device design 2284, 2284′ can be used with the same device, as shown in FIG. 22B. The rate of delivery of the drug can be controlled by varying the micro-channel design and configuration. FIG. 22C illustrates the drug eluting segment with micro-fluidic device 2280, 2280′ shown in FIG. 22A as viewed from the distal end 2270 of the drug eluting segment 2206. FIG. 22D illustrates the drug eluting segment 2206, wherein the therapeutic agent 2260 is being released from the drug eluting segment 2206, through the micro-fluidic device 2280, 2280′.

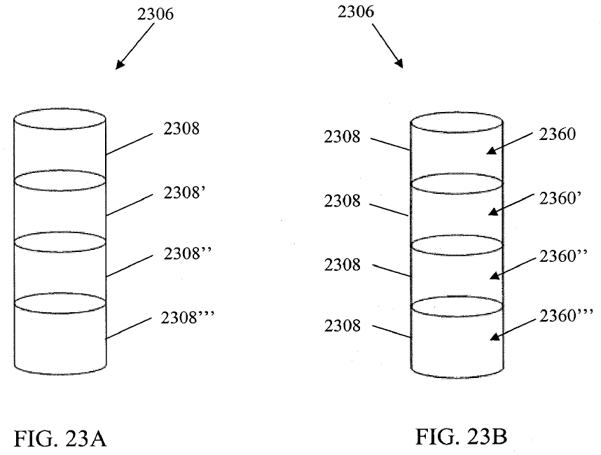

The drug eluting segment can comprise at least one drug eluting chamber. In some embodiments, the drug eluting segment 2306 can comprise more than one drug eluting chamber 2308, 2308′, 2308″, 2308′″, as shown in FIG. 23A. The drug eluting chambers can each comprise the same therapeutic agent. Alternatively, the drug eluting chambers 2308, 2308′, 2308″, 2308′″ can each comprise a different therapeutic agent 2360, 2360′, 2360″, 2360′″.

### IV. METHODS

Further provided herein are methods for closing an opening following a vitrectomy comprising obtaining access through the conjunctiva and sclera; and inserting a wound closure device into the conjunctiva and sclera, wherein the opening is formed in two or more layers of tissue, one tissue layer transposable relative to a second tissue layer. The method allows the wound closure device to be inserted into the wound without having to unnecessarily damage the surrounding tissue. The method can further comprise the step of cutting the wound closure device. In some embodiments of the method, the method can further comprise the step of positioning the conjunctive over the wound closure device. The conjunctive can be actively positioned over the wound closure device by lifting the conjunctiva over the device. Alternatively, the conjunctiva can slide passively over the wound closure device. The method can provide for a wound closure device, where the wound closure device comprises a material having a first configuration and a second configuration, wherein the device is adaptable to be inserted into a wound in the first configuration and wherein the material transitions from the first configuration to the second configuration after being inserted into the wound. In some embodiments of the method, the wound closure device is adaptable to transition between the first configuration and the second configuration after being exposed to one or more of an aqueous medium, change in temperature, change of a chemical environment, change of physical environment pH, ion strength, salt concentration, or light, or any other suitable condition to which the material is exposed. In some embodiments of the method, after the access through the conjunctiva and sclera are obtained, a cannula or any suitable structure can be inserted though the access route. Furthermore, in some embodiments, the method can further comprise the step of removing the cannula from the access route after the wound closure device has been inserted through the cannula. In some embodiments of the method, the wound closured device remains fixed in position as the cannula is being removed. Alternatively, the wound closure device can be partially retracted while the cannula is being removed. The wound closure device can be retracted at the same time the cannula is removed. Alternatively, the wound closure device can be retracted after the cannula has been removed. Additionally, the method can provide for the step of the inserting a catheter through the cannula, wherein the catheter is adaptable to facilitate the insertion of the wound closure device. The catheter can be used to insert the wound closure device into the cannula before the cannula is removed. Alternatively, the catheter can be inserted into the cannula, the cannula removed, and then the wound closure device inserted into the opening. The cannula can be inserted into the opening by pushing, blowing, or moving the wound closure device by any suitable method for positioning the device in the opening. In some embodiments of the method, the method can provide for the step of inserting a guide wire through the cannula, wherein the guide wire is adaptable to facilitate the insertion of the wound closure device. The guide wire can be used to insert the wound closure device into the cannula before the cannula is removed. Alternatively, the guide wire can be inserted into the cannula, the cannula removed, and then the wound closure device inserted into the opening. The wound closure device can be located over the guide wire. Furthermore, in some embodiments of the method, the method can further comprise the step of severing the cannula, wherein a portion of the severed cannula is adaptable to facilitate closing the wound. In some embodiments, the cannula can be severed across the top, so that the external portion of the cannula is removed from the remainder of the wound closure device. The cannula can then be filled with a suitable wound closure device. Alternatively, the exterior of the cannula that comes in contact with the opening can be coated with a biocompatible material. The interior of the cannula can be removed from the opening so that the coating remains within the opening. The interior of the coating remaining within the opening can then be filled with a suitable wound closure device. In some embodiments of the method, the wound closure device inserted is a non-solid material. In some embodiments of the method, the wound closure device inserted is a solid material. The method can further comprise the step of delivering a drug to the vitreous chamber of the eye, wherein the drug is delivered by the wound closure device.

Another method provided herein is a method for closing a wound following a vitrectomy comprising obtaining access through a portion of a conjunctiva and a sclera through a cannula; and inserting a wound closure device through the cannula, wherein the access is an opening formed in two or more layers of tissue, one tissue layer transposable relative to a second layer. Furthermore, the method can provide for the step of cutting the wound closure device after the wound closure device has been positioned in the opening. In some embodiments of the method, the method can further comprise the step of positioning the conjunctiva over the wound closure device. The conjunctiva can be actively positioned over the wound closure device by lifting the conjunctiva over the device. Alternatively, the conjunctiva can slide passively over the wound closure device. The method can further provide for the use of a wound closure device comprising a material having a first configuration and a second configuration, wherein the device is adaptable to be inserted into a wound in the first configuration and wherein the material transitions from the first configuration to the second configuration after being inserted into the wound. The material can transition from a first configuration to a first configuration to a second configuration after being exposed to one or more of an aqueous medium, change in temperature, a chemical environment, pH, ion strength, salt concentration, or light. In some embodiments, the method can provide for the step of removing the cannula after the wound closure device has been inserted through the cannula. In some embodiments, the wound closure device remains stationary in the wound as the cannula is being removed from the wound. In some embodiments, the wound closure device can be partially retracted as the cannula is removed. The wound closure device can be inserted directly into the cannula. Alternatively, a catheter can be inserted into a cannula, and the catheter used to facilitate the insertion of the device into the opening. The device can be preloaded in the catheter. Alternatively, the catheter can be inserted into the cannula and then the device loaded in the catheter. The catheter can then introduce the device into the opening. In some embodiments, the catheter is inserted into the cannula and the cannula removed. The device can then be introduced into the opening after the cannula has been removed through the catheter. The device can be pushed into the opening using a pusher rod extending through the catheter. Alternatively, the device can be drawn into the opening through capillary action. The device can be introduced into the opening using any suitable force for introducing the device into the opening. In some embodiments, the wound closure device can be introduced into an opening using a guide wire. The guide wire can be inserted into the cannula and the device introduced into the cannula using the guide wire. In some embodiments, the device is preloaded on the guide wire. In some embodiments, the guide wire is introduced into the cannula and then the device loaded on the guide wire. The guide wire can also be introduced into the cannula and then the cannula removed from the opening. The device can then be introduced to the opening using the guide wire. In some embodiments of the method, the method comprises the use of a cannula which can be used to close the wound. In such an embodiment a portion of the cannula can be used to close the wound. In some embodiments, the part of the cannula external to the eye can be severed. The remainder of the cannula can remain in the opening. The interior lumen of the cannula can then be filled with a wound closure device. Alternatively the exterior of the portion of the cannula post located within the wound can be severable from the top and interior part of the cannula post. As the cannula is withdrawn from the opening, the exterior portion of the post remains in the opening. The interior lumen of the coating can then be filled with a wound closure device. In some embodiments, the wound closure device comprises a non-solid material including, but not limited to, a gel, paste, or any other suitable non-solid material. In some embodiments, the wound closure device comprises a solid material including, but not limited to a polymer, or any other suitable biocompatible material.

Further provided herein is a method for closing an indexed wound using a wound closure device. An indexed wound comprises at least two layers of tissue, where one tissue has been transposed or displaced from its original position. The transposed tissue can be held in its displaced position during a procedure or is indexed. The method for closing an indexed wound using a wound closure device can comprise inserting a wound closure device through a wound without causing further trauma to the wound or an area surrounding the wound, the wound closure device having a first configuration and a second configuration, wherein the device is adaptable to be inserted into the wound in the first configuration and wherein the device is adaptable to transition to the second configuration after the device has be inserted into the wound.

Another embodiment of the method disclosed here is a method for closing a wound through which a procedure can be performed wherein the wound extends through at least two layers of tissue, the method comprising identifying a position of a wound; inserting a wound closure device into the wound; and closing the wound with the wound closure device, wherein the wound is formed in two or more layers of tissue, one tissue layer transposable relative to a second layer. In some embodiments, the wound is an ocular wound.

### V. KITS

Also provided herein are kits comprising the invention disclosed herein. Provided herein is a kit for closing an opening following a vitrectomy procedure comprising a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening. The kit can further comprise at least one cannula. Additionally, the kit can comprise at least one catheter. The kit can also comprise at least one guide wire. In some embodiments, at least one catheter and one guide wire can be included in the kit. In some embodiments of the kit, the wound closure device can further comprise a drug eluting segment. The drug eluting segment can be preloaded with a drug. Alternatively, the drug eluting segment can be a loadable drug eluting segment, wherein a drug is loaded into the drug eluting segment. Furthermore, the kit provided herein can further comprise at least one vial comprising at least one drug. Multiple vials may be included with the kit. In some embodiments, the multiple vials contain the same drug. In some embodiments, the multiple vials contain different drugs. An amount of one kind of drug can be introduced to the drug eluting chamber. An amount of more than one kind of drug can be introduced to the drug eluting chamber to create a drug cocktail.

Another embodiment of a kit provided herein is a kit for closing a wound following a vitrectomy procedure comprising a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening; and a plug applicator adaptable to insert the plug. The kit can further comprise at least one cannula. Additionally, the kit can comprise at least one catheter. The kit can also comprise at least one guide wire. In some embodiments, at least one catheter and one guide wire can be included in the kit. In some embodiments of the kit, the wound closure device can further comprise a drug eluting segment. The drug eluting segment can be preloaded with a drug. Alternatively, the drug eluting segment can be a loadable drug eluting segment, wherein a drug is loaded into the drug eluting segment. Furthermore, the kit provided herein can further comprise at least one vial comprising at least one drug. Multiple vials may be included with the kit. In some embodiments, the multiple vials contain the same drug. In some embodiments, the multiple vials contain different drugs. An amount of one kind of drug can be introduced to the drug eluting chamber. An amount of more than one kind of drug can be introduced to the drug eluting chamber to create a drug cocktail.

### VI. EXAMPLE

### Example 1

### Preparation of Device

In some embodiments, a collagen sheet is cut into the size 2 mm by 2 mm by 15 mm, then the cut piece is compressed in two steps such that the final cross-sectional area becomes less than 0.5 mm by 0.5 mm. The compaction is typically done at a room temperature or a temperature between 30 and 37 degree C. Then, the compressed collagen (either rectangular or circular cross-sectional shape) rod is inserted into a tubular mold, preferably Teflon tube, non-adherent polymeric or non-polymeric tubes.

Polyethylene glycol (PEG, preferably the ones with molecular weight between 1,000 and 10,000) is used as a binder. The PEG is melted at a temperature between 30 and 70 degree C., then the PEG can be sucked into the tube mold containing the compacted collagen rod. The suction can be done by vacuum, wetting by surface tension, or injection. The PEG binder solidified when the temperature drops below its melting temperature. Then, the solid-bound collagen tube is de-molded from the tube mold and the rod is ready for use.

In another embodiment, a binder can be prepared by a non-thermal method such as a solution or paste method. For example, the PEG can be mixed with a solvent (e.g. water or ethanol) to form a liquid or paste-like mixture. Then, the binder can be applied to the compacted collagen in a mold. Afterwards, the solvent evaporates and the binder solidifies in the tube mold.

In other embodiments, variety of materials can be used as a binder. These materials include, but not limited to, polyethylene glycols, any water soluble biocompatible polymers, any bioabsorbable polymers, polysaccharides such as hyaluronic acid, chondroitin sulfate, dermatan sulfate, keratan sulfate, heparan, heparin sulfate, dextran, dextran sulfate, alginate, and other long chain polysaccharides

Table 1 shows the expanding time of alternate device embodiments in distilled water. A non soluble Type 1 bovine collagen matrix was used in combination with different polymers as binder and method of application. Expanding time means the time required to expand from a firts configuration to 95% of the volume of a second configuration.

TABLE 1
Expansion of different embodiments of a collagen wound closure

| | | | | | | Expansion |
|---|---|---|---|---|---|---|
| No. | Collagen | Binder | Method | Size [mm²] | Media | Time [SEC] |
| 1 | Non Soluble | Dextrose | EtOH | 2 × 1 | distilled water | 3 |
| 2 | Non Soluble | Sucrose | Water | 2 × 1 | distilled water | 15 |
| 3 | Non Soluble | PEG2000 | EtOH | 2 × 1 | distilled water | 28 |
| 4 | Non Soluble | PEG1000 | thermal | 2 × 2 | distilled water | 98 |
| 5 | Non Soluble | PEG mixture | Thermal | 2 × 2 | distilled water | 149 |
| 6 | Non Soluble | PEG2000 | Thermal | 2 × 3 | distilled water | 126 |
| 7 | Non Soluble | PEG1450 | thermal suction | 2 × 2 | distilled water | 117 |
| 8 | Non Soluble | PEG1450 | thermal suction | 2 × 2 | rabbit vitreous | 120 |
| 9 | Non Soluble | PEG1450 | thermal suction | 2 × 2 | rabbit vitreous | 150 |

Table 2 shows the measured anchor forces of a G23 wound closure system in a rabbit eye with alternate embodiments of the wound closure. A non-soluble Type 1 bovine collagen matrix combined with different binder substances were used. Anchor forces were achieved to ensure both a stable anchoring and a minimal local tissue stress. The meaning of anchor forces is here the required force to slide the wound closure in the wound site right after the employment.

TABLE 2
Anchor force of alternate embodicments of a collagen wound
closure.

| | Collagen | Cut | | Anchor |
|---|---|---|---|---|
| No. | Type | Size [mm²] | Binder and Method | Force [g] |
| 1 | Non soluble | 2 by 2 | thermal PEG2000 | 4 |
| 2 | Non soluble | 2 by 2 | thermal PEG600/6000 | 1.5 |
| 3 | Non soluble | 2 by 2 | thermal PEG600/6000 | 2.5 |
| 4 | Non soluble | 2 by 2 | thermal PEG1450 | 1.5 |
| 5 | Non Soluble | 2 by 2 | thermal PEG1000 | 2.5 |

### Example 2

### Determination of Leakage Rates in Rabbit Eye Using Device

FIG. 18 illustrates a graph showing the leakage rates of fluid through a wound site in a rabbit eye, at different wound conditions, using one embodiment of the wound closure device described herein. The first 20 minutes just an infusion line was connected to the rabbit eye and was pressured at 35 mmHg. The leakage rate was determined by measuring the flow of the infusion line. At minute 21, a cannula was placed into the wound site and the leakage rate stabilized after about 10 minutes. The leakage rate from the open cannula was measured over 20 min. At minute 60, the cannula was plugged with the wound closure device and the leakage rate declined to base value (the value before the placement of the cannula). After applying the wound closure device, the leakage rate did not increase from the base value, which indicates sealing of the wound site. After removal of the wound closure device, the leakage rate increased rapidly to values similar to those seen with the open cannula.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A wound closure device comprising
a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening.

2. The wound closure device of claim 1 wherein the material is adaptable to transition between the first configuration and the second configuration after exposure to one or more of an aqueous medium, a change a physical environment, change in temperature, a change of a chemical environment, pH, ion strength, salt concentration, or light.

3. The wound closure device of claim 1 wherein the material is a biocompatible material.

4. The wound closure device of claim 1 wherein the material is selected from at least one of a compressible material, temperature dependent material, shape memory material, a swellable material, or an expandable material.

5. The wound closure device of claim 1 further comprising an anchor adaptable to prevent removal of the wound closure device from a wound.

6. The wound closure device of claim 1 further comprising a handle adaptable to insert the wound closure device in a wound.

7. The wound closure device of claim 1 wherein the device is adaptable to be cut.

8. The wound closure device of claim 7 wherein the device further comprises at least one marker.

9. The wound closure device of claim 1 wherein the device further comprises a drug delivery element.

10. The wound closure device of claim 1 wherein the device is adaptable to be in the first configuration after being subjected to at least one of a physical force, a chemical force, or a mechanical force.

11. The wound closure device of claim 1 wherein the device is adaptable to be inserted into a wound by a device applicator.

12. The wound closure device of claim 11 wherein the device is a pre-cut device.

13. The wound closure device of claim 11 wherein the device is adaptable to be cut by the applicator.

14. The wound closure device of claim 11 wherein the device is adaptable to be inserted through a cannula.

15. The wound closure device of claim 14 wherein the device is adaptable to be inserted into a wound while the cannula is retracted.

16. The wound closure device of claim 15 wherein the device is adaptable to be visualized as it is inserted by the applicator.

17. The wound closure device of claim 1 wherein the device is adaptable to seal a wound.

18. The wound closure device of claim 1 wherein the device is adaptable to facilitate wound in-growth.

19. The wound closure device of claim 1 wherein the device is adaptable to be inserted without relocating the opening.

20. A wound closure device for use after ocular surgery comprising
a plug adaptable to be inserted into an opening formed during ocular surgery, the opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening.

21. The wound closure device of claim 20 wherein the plug is adaptable to transition between the first configuration and the second configuration after exposure to one or more of an aqueous medium, change in temperature, a chemical environment, pH, ion strength, salt concentration, or light.

22. The wound closure device of claim 20 wherein the plug comprises a biocompatible material.

23. The wound closure device of claim 20 wherein the plug is adaptable to be in the first configuration after being subjected to at least one of a physical force, a chemical force, and a mechanical force.

24. The wound closure device of claim 20 wherein the device is adaptable to be inserted without relocating the opening.

25. A method for closing an opening following a vitrectomy comprising:
obtaining access through the conjunctiva and sclera; and
inserting a wound closure device into the conjunctiva and sclera, wherein the opening is formed in two or more layers of tissue, one tissue layer transposable relative to a second layer.

26. The method of claim 25 further comprising the step of cutting the wound closure device.

27. The method of claim 25 further comprising the step of positioning the conjunctiva over the wound closure device.

28. The method of claim 25 wherein the wound closure device comprises a material having a first configuration and a second configuration, wherein the device is adaptable to be inserted into a wound in the first configuration and wherein the material transitions from the first configuration to the second configuration after being inserted into the wound.

29. The method of claim 25 wherein the wound closure device is adaptable to transition between the first configuration and the second configuration after being exposed to one or more of an aqueous medium, change in temperature, a chemical environment, pH, ion strength, salt concentration, or light.

30. The method of claim 25 further comprising the step of inserting a cannula through the conjunctiva and sclera after the obtaining step.

31. The method of claim 30 further comprising the step of removing the cannula after the wound closure device has been inserted through the cannula.

32. The method of claim 30 wherein the removing step further comprises retracting at least a portion of the device while removing the cannula.

33. The method of claim 30 further comprising the step of inserting a catheter through the cannula, wherein the catheter is adaptable to facilitate the insertion of the wound closure device.

34. The method of claim 30 further comprising the step of inserting a guide wire through the cannula, wherein the guide wire is adaptable to facilitate the insertion of the wound closure device.

35. The method of claim 30 further comprising the step of severing the cannula, wherein a portion of the severed cannula is adaptable to facilitate closing the wound.

36. The method of claim 25 wherein the wound closure device comprises a non-solid material.

37. The method of claim 25 wherein the wound closure device comprises a solid material.

38. The method of claim 25 further comprising the step of delivering a drug to the vitreous chamber.

39. A method for closing a wound following a vitrectomy comprising
obtaining access through a portion of a conjunctiva and a sclera through a cannula; and
inserting a wound closure device through the cannula,
wherein the opening is formed in two or more layers of tissue, one tissue layer transposable relative to a second layer.

40. The method of claim 39 further comprising the step of cutting the wound closure device.

41. The method of claim 39 further comprising the step of covering the device with the conjunctiva.

42. The method of claim 39 wherein the wound closure device comprises a material having a first configuration and a second configuration, wherein the device is adaptable to be inserted into a wound in the first configuration and wherein the material transitions from the first configuration to the second configuration after being inserted into the wound.

43. The method of claim 42 wherein the device transitions from a first configuration to a second configuration after being exposed to one or more of an aqueous medium, change in temperature, a change of a physical environment, a change of a chemical environment, pH, ion strength, salt concentration, or light.

44. The method of claim 39 further comprising the step of removing the cannula after the wound closure device has been inserted through the cannula.

45. The method of claim 44 wherein the wound closure device is adaptable to be partially retracted as the cannula is removed.

46. The method of claim 39 further comprising the step of inserting a catheter through the cannula, wherein the catheter is adaptable to facilitate insertion of the device.

47. The method of claim 39 further comprising the step of inserting a guide wire through the cannula, wherein the guide wire is adaptable to facilitate insertion of the device.

48. The method of claim 39 further comprising the step of severing the cannula, wherein a portion of the severed cannula remains in the eye.

49. The method of claim 48 wherein the step of inserting the wound closure device comprises the use of the portion of the severed cannula remaining in the eye.

50. The method of claim 39 wherein the wound closure device comprises a non-solid material.

51. The method of claim 39 wherein the wound closure device comprises a solid material.

52. A method for closing an indexed wound using a wound closure device comprising
inserting a wound closure device through a wound without causing further trauma to the wound or an area surrounding the wound, the wound closure device having a first configuration and a second configuration, wherein the device is adaptable to be inserted into the wound in the first configuration and wherein the device is adaptable to transition to the second configuration after the device has be inserted into the wound.

53. A method for closing a wound through which a procedure can be performed wherein the wound extends through at least two layers of tissue, the method comprising
a. identifying a position of a wound;
b. inserting a wound closure device into the wound; and
c. closing the wound with the wound closure device,
wherein the wound is formed in two or more layers of tissue, one tissue layer transposable relative to a second layer.

54. The method of claim 53 wherein the wound is an ocular wound.

55. A kit for closing an opening following a vitrectomy procedure comprising
a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening.

56. The kit of claim 55 further comprising at least one cannula.

57. The kit of claim 55 further comprising at least one catheter.

58. The kit of claim 55 further comprising at least one guide wire.

59. The kit of claim 55 wherein the wound closure device further comprises a drug eluting segment.

60. The kit of claim 59 wherein the drug eluting segment is preloaded with a drug.

61. The kit of claim 59 wherein the drug eluting segment is a loadable drug eluting segment.

62. The kit of claim 61 further comprising at least one vial containing at least one drug.

63. A kit for closing a wound following a vitrectomy procedure comprising
a plug adaptable to be inserted into an opening formed in two or more tissue layers, one tissue layer transposable relative to a second layer, the plug comprising a material having a first configuration and a second configuration, wherein the plug is adaptable to be inserted into the opening in the first configuration and further adaptable to transition from the first configuration to the second configuration after being inserted into the opening; and
a plug applicator adaptable to insert the plug.

64. The kit of claim 63 further comprising at least one cannula.

65. The kit of claim 63 further comprising at least one catheter.

66. The kit of claim 63 further comprising at least one guide wire.

67. The kit of claim 63 wherein the wound closure device further comprises a drug eluting segment.

68. The kit of claim 67 wherein the drug eluting segment is preloaded with a drug.

69. The kit of claim 67 wherein the drug eluting segment is a loadable drug eluting segment.

70. The kit of claim 69 further comprising at least one vial containing at least one drug.
